(19) United States
(12) Patent Application Publication
(10) Pub. No.: US 2015/0290173 A1
(43) Pub. Date: Oct. 15, 2015

(54) TREATMENT OF SKIN OR MUCOSAL PATHOLOGY

(71) Applicant: ZARZATECH, INC., Cambridge, MA (US)

(72) Inventors: Sonia Divney, Boston, MA (US); Michele Di Schiena, Robecco Sul Naviglio (MI) (IT)

(73) Assignee: ZARZATECH, INC., Cambridge, MA (US)

(21) Appl. No.: 14/647,935

(22) PCT Filed: Nov. 26, 2013
(86) PCT No.: PCT/US2013/071943

§ 371 Date: May 28, 2015

Related U.S. Application Data
(60) Provisional application No. 61/790,394, filed on Mar. 15, 2013.
(60) Provisional application No. 61/847,438, filed on Jul. 17, 2013.

(30) Foreign Application Priority Data
Nov. 28, 2012 (EP) 12194594.3

(51) Int. Cl. A61K 31/4164; A61K 47/36; A61K 45/06; A61K 31/722; A61K 9/00

(52) U.S. Cl. CPC A61K 31/4164; A61K 31/722; A61K 9/006; A61K 45/06; A61K 47/36; A61K 9/0014; A61K 9/0034; A61K 9/0031

(57) ABSTRACT

The invention generally relates to compositions comprising a soluble chitosan derivative and an H2 receptor antagonist, and the use of the compositions for treating skin or mucosal diseases, such as inflammatory diseases or infectious disease.

### CROSS REFERENCE TO RELATED APPLICATIONS

This application claims the benefit of EP Application No. 12194594.3, filed Nov. 28, 2012, U.S. Provisional Application No. 61/790,394, filed Mar. 15, 2013, and U.S. Provisional Application No. 61/847,438, filed Jul. 17, 2013. All of the foregoing patent applications are incorporated herein by reference in their entireties.

### FIELD OF THE INVENTION

The invention generally relates to compositions comprising a soluble chitosan derivative and an H2 receptor antagonist, and the use of the compositions for treating or preventing progression of pathology of the skin or mucus membranes, such as inflammatory diseases or infectious disease.

### BACKGROUND OF THE INVENTION

Periodontal disease is a group of diseases affecting the periodontium. Any inherited or acquired disorder of the tissues surrounding and supporting the teeth (periodotium) can be defined as a periodontal disease, but the term usually refers to the common inflammatory disorders of gingivitis and periodontitis that are caused by pathogenic microflora in the biofilm or dental plaque that forms adjacent to the teeth on a daily basis. The inflammation associated with periodontal disease is caused by the host response to specific microorganisms. Tissue and gingival crevicular fluid levels of histamine are reported to be increased in patients with gingivitis and periodontitis. Histamine alters a variety of neutrophil, macrophage, and monocyte functions mediated through the binding of H2 receptors on the cell surface. Schenkein, H., The Pathogenesis of Periodontal Diseases, Academy Reports, Journal of Periodontology, 457-466 (1999).

Because periodontitis is a chronic inflammatory disease that can be caused by injury or bacteria, it follows that two mechanisms of treating periodontal disease would be to control either the bacterial infection or to control the inflammatory or so-called host-factor related aspect of the disease.

The mechanical removal of the microbial biofilm has been the standard for prevention and treatment of periodontal conditions since ancient times. Although the microbial plaque is necessary for gingivitis and periodontitis to occur, it is not sufficient on its own to cause periodontitis. Differences in host response to the microbial biofilm appear to be the critical determinant of disease severity, extent and progression. The difference in host response is the reason why, in the absence of plaque control, a patient will develop mild versus severe gingivitis, and further, whether or not the presence of a chronic plaque burden causes gingivitis to progress to periodontitis. Reinforcement of the leukocyte barrier against oral pathogens is critical to preventing both the transition from gingivitis to periodontitis and periodontal disease progression. Attstrom, R., The Roles of Gingival Epithelium and Phagocytosing Leukocytes in Gingival Defence, Journal of Clinical Periodontology, 2:25-32 (1975).

Cimetidine has been used in gastroenterology in the treatment of benign gastric and duodenal ulcers, reflux esophagitis, Zollinger-Ellison syndrome, systemic mastocytosis, and multiple endocrine adenomas. Cimetidine is a selective antagonist of the H2 receptors and inhibits the histamine stimulated release of gastric acid (thereby reducing the secretion of gastric acid), and is therefore used widely for the treatment of peptic ulcer. Kenyon, G. S., et al., Cimetidine and the Gastric Mucosal Barrier, Gut, 18:631-635 (1977). Cimetidine eliminates the downstream inhibitory actions of histamine on chemotaxis, phagocytosis, superoxide anion production and the production of TNF-α and IL-12 by macrophages.

Cimetidine has been investigated for its potential use in treatment of pathologies of the oral cavity, in particular of periodontium pathologies characterized by inflammation and pain. See, e.g., US 2008/0045575 A1, U.S. Pat. Nos. 5,294,433 and 5,364,616. For example, cimetidine has been formulated as topical pharmaceutical formulation, a mouth-wash, for the treatment of pathologies of the oral cavity, in particular of periodontium pathologies. However, the activity of cimetidine was reduced when it was topically administered to the oral cavity in humans, and this reduced activity was further negatively affected by its low water solubility, and the detergent action of the saliva.

Some in vivo studies indicated that cimetidine reduced gingivitis in a dog gingivitis model. Gao, C., et al., Effects of Cimetidine on Progression of Naturally Occurring Periodontitis in Beagle Dogs (2002) (Abstract available at http://iadr.confex.com/iadr/2002SanDiego/techprogram/abstract_{—}11483.htm). These studies demonstrated that cimetidine significantly reduced gingival inflammation and gingival bleeding with no antiplaque activity or increase in tooth stain. See, Snider et al. (Evaluation of H2-Receptor Antagonists—Cimetidine, Ranitidine and Famotidine in an In-vivo Gingivitis Model; Research presented at the 80th General Session of the IADR, Mar. 6-9, 2002; available at www.dentalcare.com/media/en-US/journals/pgresrch/posters/iadr02/pdfs/859.pdf, or http://iadr.confex.com/iadr/2002SanDiego/techprogram/abstract11483.htm). However, Snider also reports that plaque and bacterial control were not achieved by administration of an H2 antagonist. Consequently, the prevention of gingivitis and disease progression was minimal when compared to other standard treatments such as scaling and root planning Bleeding and probing and clinical inflammation was slightly reduced, but not significant enough to be considered as an option in prevention and treatment of periodontal disease progression.

In a dose response study in the same dog gingivitis model, it was shown that cimetidine HCL had significant anti-gingivitis activity that increased with increasing dose. However, in a prevention study in the same dog model, results were not conclusive. In a series of clinical studies in humans that evaluated a topical cimetidine rinse on neutrophil function in the gingival crevice, it was shown that topical 0.5% cimetidine oral rinse enhanced antibacterial function of crevicular neutrophils, but with no clinical benefit on gingivitis.

Dental plaque is a complex microbial community growing as a biofilm on enamel surfaces. Several studies have been conducted on early colonizers within in vivo dental biofilm, and to establish potential population shifts that occur during the early phases of biofilm formation. See, e.g., Li, E. J. et al., Identification of early microbial colonizers in human dental biofilm, J. Appl. Microbiol., 97(6):1311-8 (2004). However, it is difficult to draw cause and effect inferences from these studies.

One implication from studies on biofilm is that periodontitis is an inflammatory disease, and the primary target of pharmacotherapy should be the initial superficial inflammation, rather than the bacteria localized in deep pockets. Consequently, standard treatment involving inserting therapeutics into periodontal pockets has been re-evaluated. Early colonizers of the periodontal pockets cannot be removed from the pockets as they are part of the natural flora of the mouth. Inflammation should be targeted at the surface of the mucosa before reaching the pockets. See, e.g., Ekstein, J., Shapira, L., Van Dyke, T. E. “The pathogenesis of periodontal disease: a paradigm shift,” Refuat Hapeh Vehashinayim. 27(3):35-9, 63 (July 2010).

Selective H2 antagonists are not generally known as anti-inflammatory agents but surprisingly act to reduce inflammation. There are several problems, however, with oral topical application of H2 antagonists to the mucosal tissues of the oral cavity as described in the U.S. Pat. No. 5,294,433. First, these chemicals are not well absorbed by the gingival tissues. Second, saliva washes away the chemicals, thereby reducing their therapeutic effect.

Due to their poor absorption and the slight damage they cause to the membrane barrier, H2 antagonists can take many weeks, or even months of treatment, to have a therapeutic effect on periodontal disease. This has been demonstrated through extensive study of the use of H2 antagonists for the treatment of stomach ulcers, and in gingivitis models. This effect is primarily due to the modulation of the immune system over time by the H2 antagonist, and is not due to the immediate topical effects of the compound. H2 antagonists alone do not enhance the membrane stability and mucosa of the mouth to prevent or treat damage to the tissues. Topically applied H2 antagonists have also been shown to cause irritation and cell death around permeable tissues in the mouth. Finally, as explained earlier, H2 antagonists do not have enough antibacterial effect, which is needed in order to protect the mucosa.

Improved formulations of H2 antagonists that can be administered topically to the oral mucosa are needed.

Chitosan, a cationic copolymer of glucosamine and N-acetyl-D-glucosamine, is a partially deacetylated derivative of a natural polysaccharide, chitin. While chitosan and its derivatives are used in many applications, including pharmaceutical, its use is severely limited because it is insoluble at neutral and alkaline pH. Solubility is only observed below pH 6.5, which is the pKa of chitosan. Mourya, V. K. et al., Carboxymethyl Chitosan and its Applications, Advanced Materials Letters, 1(1), 11-33 (2010). Chitosan has bioadhesive properties and has been shown to adhere to the epithelial tissues and to the mucus coat present on the surface of the tissues. Bansal, V. et al., Applications of Chitosan and Chitosan Derivatives in Drug Delivery, Advances in Biological Research, 5(1):28-37 (2011).

Chitosan is thought to have potential as an agent for controlled release drug delivery because of its biocompatibility, biodegradability, bioactivity, and nontoxicity. However, again, a significant drawback to the use of chitosan for these purposes remains its insolubility in water. Furthermore, chitosan has limited capacity for controlled the release of an encapsulated compound and requires chemical crosslinking in order to avoid rapid dissolution of the encapsulated compounds into the gastric cavity for peroral formulations. Other investigators have made cellulose/chitosan microspheres in order to solve some of these problems. These microspheres were shown to adhere to the gastric mucous layer and have potential for controlled drug release, however the cellulose comprised the outer layer of the microspheres. On the other hand, using H2 receptor antagonist in the outer layer of the microspheres could reduce the therapeutic effect, if applied to oral mucosa, as the H2 receptor antagonist would be diluted. Zhou, H., et al, In Vivo and In Vitro Evaluation of Mucoadhensiveness [sic] of Chitosan/Cellulose Acetate Microspheres, Journal of Biomedical Materials Research, 1146-1153 (2007).

Chitosan has been used topically to treat periodontal disease. Chitosan rinses have been shown to be effective in reducing plaque formation and counts of salivary mutans streptococci after a 14-day rinsing period. Mutans streptococci are a group of oral streptococci that are closely related to Streptococcus mutans. Streptococcus mutans is a facultative anaerobic, Gram positive coccus-shaped bacterium that is commonly found in the human oral cavity and is a significant contributor to tooth decay. Despite the fact that chitosan showed initial promise as an effective anti-plaque agent for use in oral hygiene products, there are a number of issues with delivering the chitosan to the oral tissues in an effective manner. Chitosan washes away easily from oral tissues. Chitosan is poorly soluble in water. Low molecular weight forms of chitosan with a molecular mass of 5-6 k Da and a degree of deacetylation of between about 50% and 60% are more effective than high molecular weight chitosans (low molecular weight chitosan typically refers to chitosan having a molecular weight up to about 50,000 Dalton). See, Sano, H., et al., Effect of Molecular Mass and degree of Deacetylation of chitosan on adsorption of Streptococcus sobrinus 6715 to saliva treated hydroxyapatite, Bull Tokyo Dent Coll., 2002 43(2):75-82. Chitosan rinses have also been shown to increase the permeability of the oral mucosa reducing the membrane stability and resistance to recovery. The same has been found for chitosan derivatives such as N,O-carboxymethyl-chitosan.

In light of the difficulties in delivering H2 antagonists and chitosan in a manner that results in therapeutic effect, there is a need in the art for compositions that are capable of doing so. Previous attempts at remedying this problem have focused on liposomal delivery of these active agents, as described in U.S. Patent Publication No. US 2011/0135716 A1. However, further investigation showed that these liposomal formulations were unsuitable for use as topical oral agents. These liposomal formulations had very poor shelf stability, required special storage conditions, and could not be mixed with the standard other components typical of topical oral products, such as flavorings, preservatives, anti-bacterial agents (such as low weight Chitosan or chitosan derivatives) and other active ingredients of interest. Thus, these formulations are essentially useless as a therapeutic of interest for humans.

As a result, there is a need in the art for stable formulations that are suitable for topical oral administration and that enhance the delivery, effectiveness and life cycle of a drug, preferably an H2 antagonist.

### BRIEF SUMMARY OF THE INVENTION

The present invention relates to compositions comprising a host modulator, such as a H2 receptor antagonist (e.g., cimetidine), and a water soluble chitosan derivative (e.g., N,O-carboxymethyl chitosan).

The compositions of the invention are particularly useful for the prevention and treatment (including delaying the progression) of oral or dental pathologies characterized by inflammation and pain, in particular for the prevention and treatment (including delaying the progression) of pathologies of the periodontium. The compositions of the invention are suitable for human and veterinary uses.

Novel compositions comprising a formulation with a mucosal-adhesive polymer N,O-carboxymethyl-chitosan in combination with an H2 antagonist, preferably cimetidine, having an electrical resistance associated with the integrity of the tight junctions are disclosed herein. Also disclosed are methods of using the compositions, for the treatment of inflammatory and/or infectious disease, including psoriasis and cold sores, as well as oral infectious disease, especially gingivitis and periodontitis, and pathology around implants (medical and/or dental implants).

These compositions exhibit unexpected mucoadhesive properties and can therefore be used to treat or prevent periodontal disease progression by protecting and maintaining the barrier function of the oral gingival tissues against plaque pathogens or injury of the oral mucosa. The composition further forms a protective film over the oral gingival tissues.

The mucoadhesive action or film forming ability of the compositions is promoted and further increased by the presence of water. In some embodiments the composition is a water based mouthwash. In other embodiments, the composition does not comprise alcohol. In some formulations, little or no water is present. The physiological water present in the oral cavity ensures an increase in mucoadhesivity for such compositions.

The compositions can be used to treat, including preventing the progression of, skin and mucosal pathologies as described herein, in particular oral pathologies such as oral infectious or inflammatory diseases including gingivitis, periodontitis, mucositis and peri-implantitis. The invention also relates to methods of treating or preventing progression of (including delaying the progression) skin and mucosal pathologies as described herein, by administering an effective amount of the composition to a subject in need thereof.

### BRIEF DESCRIPTION OF THE DRAWINGS

FIG. 1 is an illustration of morphology and function of tight junctions (Sawada et al., (2003) Med Electron Microsc. 36: 147-56).

FIG. 2 is chart showing change in TEER after exposure to Formulation No. 3.

FIG. 3a shows the absolute TEER values after 10 min exposure to the test formulations. FIG. 3b shows the normalized TEER values after 10 min exposure to the test formulations.

FIG. 4 shows the release of LDH in the medium underneath after 10 minutes of exposure to the test formulations.

FIG. 5 shows the results of Lucifer Yellow permeability test after 10 minutes of exposure to the test formulations (30 minutes and 2 hours are the time points of LY assay recording).

FIG. 6 is an LDH standard curve.

FIG. 7 is a graphical depiction of LY assay.

FIG. 8a shows TEER measurement at 1 hour recovery of injured tissues (duplicate tissues). FIG. 8b shows TEER measurement at 1 hour recovery of non-injured tissues (duplicate tissues).

FIG. 9a shows TEER measurement at 6 hour recovery of injured tissues (duplicate tissues). FIG. 9b shows TEER measurement at 6 hour recovery of non-injured tissues (duplicate tissues).

FIG. 10a show the release of LDH after 10 min+1 h/6 h from injured tissues (duplicate tissues). FIG. 10b show the release of LDH after 10 min+1 h/6 h from non-injured tissues (duplicate tissues).

FIG. 11a shows the results of lucifer yellow paracellular flux assay after 10 min+1 h recovery of non-injured tissues. FIG. 11b shows the results of lucifer yellow paracellular flux assay after 10 min+6 h recovery of non-injured tissues.

FIG. 12a shows the results of lucifer yellow paracellular flux assay after 10 min+1 h recovery of injured tissues. FIG. 12b shows the results of lucifer yellow paracellular flux assay after 10 min+6 h recovery of injured tissues.

FIG. 13 shows the instrument for TEER measurement.

### DETAILED DESCRIPTION

The present invention relates to compositions comprising a host modulator, such as a H2 receptor antagonist (e.g., cimetidine), and a water soluble chitosan derivative (e.g., N,O-carboxymethyl chitosan).

In one aspect, the invention provides a novel composition comprising a mucosal-adhesive polymer, N,O-carboxymethyl-chitosan, in combination with an H2 antagonist (preferably cimetidine), having an electrical resistance associated with the integrity of the tight junctions in soft tissue.

The invention also provides a method of using the compositions described herein (e.g., a composition comprising a water soluble chitosan derivative and an H2 receptor antagonist), for the treatment of inflammatory diseases (for example, inflammation around a medical implantable device and a dental implant, such as mucositis and peri-implantitis), as well as oral infectious diseases, and the use of the compositions for treating or preventing pathology of the skin or mucus membranes, such as periodontal inflammatory diseases or infectious diseases. Such treatment includes delaying the progression the disease.

1. Introduction—Barrier Function of Oral Gingival Tissues

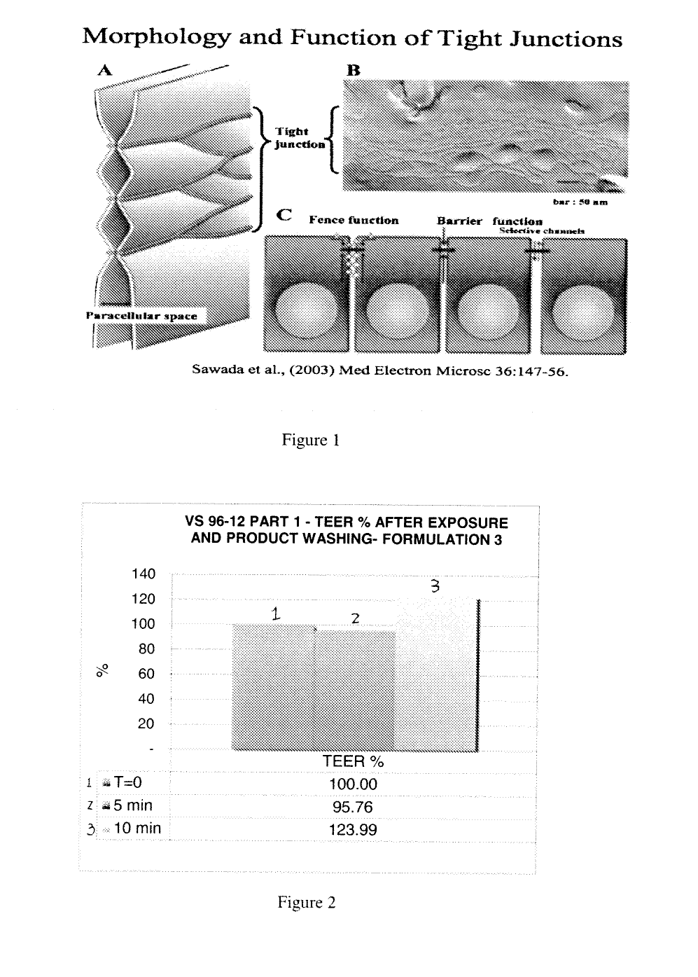

It is important to protect and maintain the barrier function of oral gingival tissues. The compositions of the instant invention maintain the healthy function of the oral mucosal tissue with an increase of epithelial membrane stability, as measured by trans-epithelial-electrical-resistance (“TEER”). TEER is the measure of the movement of ions across the paracellular pathway regulated by polarized plasma membrane surfaces and by cell-to-cell tight junctions that together prevent movement of solutes and ions across the epithelia. See FIG. 1.

TEER is an indirect assessment of tight junction stability and consequently is a direct measure of the barrier function in epithelial tissue. TEER reflects the global resistance of the barrier linked both to the structure and to epithelial thickness.

Maintenance of stability and electrical resistance of the epithelium is important for normal physiological processes, therefore significant changes in TEER may represent an early expression of cell damage and it can be considered a complementary parameter.

The consequence of maintaining this barrier function is the prevention of new or existing disease from further progression and thereby maintaining the integrity of periodontal tissues, thereby preventing or treating periodontitis.

According to Shojaei, A. H., et al., Buccal mucosa as a route for systemic drug Delivery, J. Pharm. Pharm. Sci., 1:15-30 and (1998) Sohi, H., et al., Critical Evaluation of Permeation Enhancers for Oral Mucosal Drug Delivery, Drug Development and Pharmacy, 36(3): 254-282 (2010), oral mucosa is a “leaky” epithelium, intermediate between that of the relatively impermeable epidermis of skin and the highly permeable intestinal mucosa. Keratinized epithelia (e.g., gingival and hard palate) are relatively impermeable to water and show barrier function. The barrier function is due to the presence of neutral lipids such as ceramides and acylceramides. Nonkeratinized epithelia (e.g., buccal, sublingual and soft palatal) do not contain acylceramides and only have small amounts of ceramides. They also contain small amounts of neutral but polar lipids, mainly cholesterol sulfate and glucosyl ceramides. These epithelia are found to be more permeable to water than keratinized epithelia. Keratinized and nonkeratinized tissues occupy about 50% and 30%, respectively, of the total surface area of the mouth.

Permeability is inversely proportional to the degree of keratinization and relative thickness of the tissue. It is estimated that the permeability of the buccal mucosa is 4-4000 times greater than that of skin. Small molecules (<75-100 Da) are able to cross the oral mucosa rapidly. The oral mucosa is made up of the epithelium and the lamina propria, a thin layer of loose connective tissue which lies beneath the epithelium. There is an inverse relationship between permeability and molecular size mechanical barrier, protecting underlying tissues, whereas the lamina propria acts as a mechanical support and also carries blood vessels and nerves.

Drug delivery via oral mucosa is an alternative method of systemic administration for various classes of therapeutic agents. Among the oral mucosae, buccal and sublingual mucosae are the primary focus for drug delivery. Buccal delivery offers a clear advantage over the peroral route due to the avoidance of the gastrointestinal tract and hepatic first-pass metabolism. However, despite offering the possibility of improved systemic drug delivery, buccal administration has been utilized for relatively few pharmaceutical products so far. One of the major limitations associated with buccal delivery is low permeation of therapeutic agents across the mucosa. Sohi, H., et al., Critical Evaluation of Permeation Enhancers for Oral Mucosal Drug Delivery, Drug Development and Pharmacy, 36(3): 254-282 (2010).

It is very important to ensure that the effect of permeation enhancer on membrane permeability is not permanent and that the membrane should revert to its normal integrity and barrier function upon removal of the enhancer. It has been shown that ionic surfactants that are commonly found in toothpastes and other oral medicaments (sodium lauryl sulfate and cetyl pyridiniumchloride) can damage and separate the keratinized layers of epithelium, with loss of surface squames when applied to the ventral surface of the tongues of dogs. Sodium deoxycholate (0.5%) or sodium lauryl sulfate (0.1%) when applied to rabbit buccal mucosa showed loss of surface epithelial layers. Sohi, H., et al., Critical Evaluation of Permeation Enhancers for Oral Mucosal Drug Delivery, Drug Development and Pharmacy, 36(3): 254-282 (2010); Gandhi R. B. & Robinson J R., Mechanism of penetration enhancement for transbuccal delivery of salicylic acid. Int J Pharm, 85:129-40 (1992).

The outer epithelium is considered to be the rate-limiting membrane to mucosal permeation. This barrier exists in the outermost (200 μm) superficial layer of the oral mucosa (i.e., uppermost 20-30% of epithelial layer). Dowty, M. E., et al., Transport of thyrotropin releasing hormone (TRH) in rabbit buccal mucosa in vitro. Pharm Res, 9:1113-22 (1992); Gandhi R. B. & Robinson J R., Mechanism of penetration enhancement for transbuccal delivery of salicylic acid. Int J Pharm, 85:129-40 (1992). Squier and his co-workers performed a series of permeation studies using large molecular weight tracers, such as lanthanum nitrate and horseradish peroxidase to understand the barrier functions of epithelium. When applied to the outer surface of epithelium, these tracers penetrate only through the outermost two or three layers of cells. When applied to the subepithelial region, they permeate through the connective tissue, the basal lamina, and through the lower 75% of intercellular spaces of the epithelium but not into the outermost 25% cell layers of the epithelium. This suggested that flattened surface cell layers, which are present in upper one third of the epithelium, present the main barrier to permeation, whereas the more isodiametric cell layers are relatively permeable. The basement membrane is a continuous membrane having thickness of approximately 1 μm. It also presents some hindrance to permeation of proteins, immune complexes, endotoxins, and certain therapeutic agents such as chlorhexidine and beta blockers. Squier C A, Rooney L. The permeability of keratinized and non-keratinized epithelium to lanthanum in vivo. J Ultrastruct Res, 54:286-95 (1976); Hill M W, Squier C A. The permeability of rat palatal mucosa maintained in organ culture. J Anat, 128:169-78 (1979); Squier C A, Hall B K. The permeability of skin and oral mucosa to water and horseradish peroxidase as related to the thickness of the permeability barrier. J Invest Dermatol, 84:176-9 (1985); Squier C A, Hall B K. The permeability of mammalian non-keratinized oral epithelia to horseradish peroxidase applied in vivo and in vitro. Arch Oral Biol, 29, 45-50 (1986).

Various substances have been explored as permeation enhancers to increase the flux/absorption of drugs through the mucosa, but irritation, membrane damage, and toxicity are always associated with them and limit their use. A clinically acceptable permeation enhancer must increase membrane permeability without causing toxicity and permanent membrane damage. However, optimizing the concentration of enhancer to limit its toxicity while facilitating an enhancing effect reproducibly has been very challenging. There remains a need in the art for compositions that can safely and effectively deliver drugs. The compositions and methods disclosed herein fulfill this need. The instant combination products allow the treatment of oral diseases with a drug permeation enhancer that is rapidly reversible in action and does not cause irreversible toxic and damaging effects to the membrane (i.e., selective only against the target cells and inert with respect to cells participating in irritation).

An “effective” composition is one that is capable of delivering the active ingredients of the composition to the affected areas of the mucosa and acts to treat or prevent periodontal disease (including e.g., delaying the progression, ameliorate the severity, or reducing the incidences of periodontal diseases). A “safe” composition is one that protects and maintaining the barrier function.

II. Compositions

The compositions comprise a host modulator, such as a H2 receptor antagonist (e.g., cimetidine), a mucoadhesive antibacterial agent, such as a water soluble chitosan derivative (e.g., N,O-carboxymethyl chitosan), and a carrier and excipients. Preferably, the composition comprises a mucosal-adhesive chitosan derivative and an H2 antagonist.

As described above, the prior art shows numerous issues which, until now, rendered the administration of H2 antagonists for the purpose of treating periodontal pathologies ineffective. The invention is based, in part, on the discovery that the addition of a chitosan soluble derivative to topical compositions of histamine H2 receptor antagonists is advantageous in that it gives the compositions surprising mucoadhesive properties and improves efficacy.

Water soluble chitosan derivatives are well-known in the art. For example, water soluble chitosan derivatives with a range of molecular masses and degrees of deacylation that adsorb bacteria have been described in Sano et al., Bull. Tokyo dent. Coll., 43(2)75-82 (2002).

Unexpectedly, combining an H2 antagonist with a water soluble chitosan derivative results in surprising mucoadhesive properties. The compositions are capable of forming a protective film over the muscosal tissues, resulting in both the stabilization and protection of the tissue while also enabling the H2 antagonist to have longer lasting effect. The film formed resists the washing away by saliva observed with the prior art compositions. Furthermore, the compositions have an immediate protective effect (within 10 minutes) on the mucosal tissue, which lasts for long time (at least six hours).

The immediate protection provided by the water soluble chitosan derivative provides stability to the mucosal tissue. Due to this stability, the H2 antagonist is able to contribute its immune boosting effects far more quickly than observed with the prior art formulations.

Accordingly, the disclosed compositions preferably comprise a host modulator and a mucoadhesive antibacterial agent. In some embodiments the host modulator is an H2 antagonist. Non-limiting examples of suitable H2 antagonists are cimetidine, ranitidine, famotidine, and nizatidine. In addition to the therapy of gastric and duodenal ulcers and other gastro-intestinal diseases characterized by acid hyper-secretion, these H2 antagonists can also be used for inflammatory diseases of the mucous membranes. In preferred embodiments, the H2 antagonist is cimetidine.

In certain embodiments, the concentration of the H2 antagonist ranges between about 0.01% and about 10%, preferably between about 0.1% w/w and about 5% w/w.

In certain embodiments, the concentration of the H2 antagonist ranges between about 0.001% and about 10%, preferably between about 0.01% w/w and about 5% w/w.

A number of classes of compounds have been evaluated as host modulation agents. In some embodiments, the host modulator is selected from the classes of drugs including nonsteroidal antiinflammatory drugs (NSAIDs), bisphosphonates, tetracyclines, cytokine antagonists, nitric oxide synthase inhibitors, enamel matrix tetracyclines, growth factors and bone morphogenetic proteins.

Non-limiting examples of suitable mucoadhesive antibacterial agents are water soluble chitosan derivatives. Chitosan itself is poorly water soluble; therefore other compounds of interest were examined for suitability for administration. Carboxymethylation of chitosan has been shown to improve its solubility in water. Carboxymethyl-chitosan has also been shown to be non-toxic, and has the potential to have antioxidant, antibacterial, and anti-apoptotic activity. Mourya, V K et al., Carboxymethyl Chitosan and its Applications, Advanced Materials Letters 1(1), 11-33 (2010). N,O-carboxymethyl-chitosan has been shown to prevent postoperative peritoneal adhesions, without impeding normal healing. Kennedy, R., Prevention of Experimental Postoperative Peritoneal Adhesions by N,O-carboxymethyl-chitosan, Surgery, 120(5): 866-870 (1996); International Patent Application No. PCT/US98/09001.

As disclosed and exemplified herein, it was unexpectedly found that chitosan derivatives that are more water soluble, for example, N,O-carboxymethyl-chitosan, exhibit surprising mucoadhesive properties which allow it to form a film over the tissue to which it is topically applied. Also surprising was that combining the water soluble chitosan derivative with an H2 antagonist results in the chitosan derivative acting as a carrier and/or enhancer for the H2 antagonist.

In certain embodiments, the chitosan soluble derivative comprises a carboxy or sulfonic group, preferably a carboxy, ionizable group. An example of chitosan soluble derivative according to the invention is N,O-Carboxymethyl chitosan, whose preparation is reported in U.S. Pat. No. 4,619,995.

N,O-carboxymethyl chitosan, is commercially available (for example from HEPPE MEDICAL CHITOSAN GmbH under the trade mark Chitoceuticals®), has useful properties in the dental field, in particular hydrating and/or film-forming actions, protective action of the mucous membranes against pathogenic agents, and buffering action on the oral cavity physiological pH.

In certain embodiments, the concentration of the water soluble chitosan derivative (e.g., N,O-carboxymethyl chitosan) ranges between about 0.001% and about 10%, and is preferably between about 0.01% w/w and about 5% w/w.

The novel compositions disclosed herein maintain periodontal health in patients with periodontal disease, as reflected in increased bone density, pocket reduction, and decreased signs of clinical inflammation. The compositions act in a protective capacity, even in the absence of optimal oral hygiene. This allows the compositions to be used as standalone therapies that do not have to be strictly accompanied by mechanical debridement of affected tissues.

Exemplary embodiments of the compositions of the invention also have the following further advantageous aspects: a) stability in a wide pH range, between 4 and 8; b) stability to thermal changes in a wide range of temperatures, in particular between −10° C. and +40° C.; c) stability to a number of ingredients (active principles and excipients) conventionally used in the topical preparations; and d) stability to dilution, even high, with water.

The compositions can be formulated for topical administration, e.g. topical oral administration. In certain embodiments, the formulations are in the form of mouth-washes, gel, spray, foams, emulsions, dentifrices, for use in the treatment of affections of the oral mucous membranes (periodontitis and the like), or in the form of gel, ointments, creams, powders, patches and transdermal forms for use in the treatment of lesions and wounds of the skin or of rectal and vaginal mucous membranes.

The disclosed novel compositions are suitable for formulating for standard oral applications because they can be combined with excipients including, but not limited to, flavorings, preservatives, and other active ingredients, including, but not limited to, nutrients, vitamins, omega-3 fatty acids, hyalauronic acid, disinfectants of the oral cavity, steroidal or non-steroidal anti-inflammatories, wound healing agents, analgesics, antimicrobials, and antihistamines. Flavorings are particularly preferred where the composition would otherwise have a bad taste.

Excipients or carriers used in the compositions of the invention should be compatible with the H2 antagonist (e.g., cimetidine) and with the chitosan derivative. Conventional excipients and carriers which may be of use are described in the Handbook of Pharmaceutical Excipients, 6^{th} Edition, Pharmaceutical Press, the contents of which are incorporated herein. Preferably, the compositions are formulated for topical administration.

Physiologically acceptable carriers or excipients for use with the inventive compositions can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, emollients, and skin protecting agents.

Examples of solvents are water, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a composition of the invention to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin; sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carrageenans, hyaluronates, and alginates. Ointment bases suitable for use in the compositions of the present invention may be hydrophobic or hydrophilic, and include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

The pharmaceutical compositions of the invention may, alternatively or additionally, comprise other types of excipients including, thickening agents, bioadhesive polymers, and permeation enhancing agents.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. Particularly interesting are thickening agents with thixotropic properties (i.e., agents whose viscosity is decreased by shaking or stirring). The presence of such an agent in a pharmaceutical composition allows the viscosity of the composition to be reduced at the time of administration to facilitate its application to the site of interest (e.g., to the gingiva or periodontal pocket) and, to increase after application so that the composition remains at the site of administration.

In embodiments where an inventive pharmaceutical composition is intended to be applied on skin, bioadhesive polymers are useful to hydrate the skin and enhance its permeability. Bioadhesive polymers can also function as thickening agents. Examples of bioadhesive polymers include, but are not limited to, pectin, alginic acid, chitosan, polysorbates, polyethylene glycol), oligosaccharides and polysaccharides, cellulose esters and cellulose ethers, and modified cellulose polymers. Permeation enhancing agents are vehicles containing specific agents that affect the delivery of active components through the skin. Permeation enhancing agents include solvents, such as alcohols (e.g., ethyl alcohol, isopropyl alcohol), dimethyl formamide, dimethyl sulfoxide, 1-dodecylazocyloheptan-2-one, N-decylmethylsulfoxide, lactic acid, N,N-diethyl-m-toluamide, N-methylpyrrolidone, nonane, oleic acid, petrolatum, polyethylene glycol, propylene glycol, salicylic acid, urea, terpenes, and trichloroethanol) and surface active compounds.

In embodiments where an inventive pharmaceutical composition is intended to be applied on skin, the pharmaceutical composition may be packaged as kits comprising a container including the composition, optionally admixed with physiologically acceptable carriers or excipients, and at least one dressing, wherein the dressing is to be applied to cover the skin site following local administration of the content of the container to the site. The term “dressing” refers to any covering designed to protect a skin site. The term includes porous and non-porous coverings, woven and non-woven coverings, absorbent coverings, and occlusive coverings. The dressing may also be used as a delivery system for the pharmaceutical composition of the invention. For example, the pharmaceutical composition may be incorporated into or coated onto the dressing (e.g., by dipping the dressing in or spraying the dressing with the pharmaceutical composition of the invention).

In embodiments where an inventive pharmaceutical composition is intended to be administered to the oral cavity, the composition may desirably comprise other components, such as, for example, topical oral carriers. Such carriers include, but are not limited to, anticaries agents, antiplaque agents, anticalculus agents, anti-inflammatory agents, dental abrasives, flavoring agents, sweetening agents, binders, humectants, thickening agents, buffering agents, preservatives, coloring agents, and pigments, flavorants, fillers, stabilizers, ethanol and water.

In view of the instant disclosure, the compositions of the invention can be prepared according to known methods, described for example in Remington, The Science and Practice of Pharmacy, 20th Edition, typically in forms useful for the treatment of afflictions of the oral mucous membranes (periodontitis and the like).

The compositions are effective in protecting the oral tissues while ensuring effective, therapeutic delivery of the active ingredients.

Mucoadhesivity allows for Cimetidine or the other antagonists H2 to remain for a longer time on mucous membranes, in particular on the oral mucous membranes, thus enhancing the penetration of the medicament with evident, advantageous therapeutic benefits. The mucoadhesive action of the compositions is promoted and further increased by the presence of water; water based mouth-washes are therefore particularly preferred. The physiological water present in the oral cavity also ensures an increase in mucoadhesivity even to compositions with very low—if any—water content. Another advantage of these compositions is that they may be alcohol free. Presently, most of the compositions available in liquid form to treat periodontal disease require the inclusion of at least some alcohol for its antiseptic and antibacterial properties

The compositions of the invention can further comprise other active agents useful for the topical treatment of oral mucous membranes, described for example in Martindale, The Complete Drug Reference, 34^{th} Edition. Examples of said further active principles comprise disinfectants of the oral cavity such as Propolis, chlorhexidine, benzalkonium, cetylpyridinium, Triclosan, silver and derivatives; steroidal or non steroidal anti-inflammatories such as Cortisone emisuccinate, Diclofenac, Ibuprofen, Ketoprofen; wound-healing agents such as Aloe vera, allantoin and derivatives, Liquorice and derivatives; analgesics such as Lidocaine and Benzydamine; antimicrobials, antihistamines.

Other suitable active agents include, e.g., Omega 3 fatty acids, lipoxins, resolvins, hyaluronic acid, and herb extract or derivatives such as extracts from Centella asiatica, Echinacea purpurea, and Sambucus nigra.

Unlike the prior art liposomal delivery systems (US 2008/045575A1), which were highly unstable, the compositions described herein are stable over a wide range of temperatures, between about −10° C. and about +40° C. and pH, between about 4 and about 8.

The compositions are stable even when combined with other ingredients (e.g., another active ingredient, a formulation excipient), including ingredients that are conventionally used in topical preparations, for example, ingredients described in Martindale, the complete drug reference, 34 edition. The compositions are also stable even when greatly diluted with water or scaled up. Unlike the prior art liposomal delivery systems, the compositions disclosed herein may be formulated into a variety of products suitable for human uses. Depending on the mode of administration, the inventive pharmaceutical compositions may be in the form of liquid, solid, or semi-solid dosage preparation. Examples include, but are not limited to, solutions, mouth-washes, dispersion, suspensions, emulsions, mixtures, lotions, liniments, gels, jellies, ointments, creams, pastes including toothpastes, dentifrices, gels, hydrogels, aerosols, sprays including mouth sprays, powders including tooth powders, granules, granulates, lozenges, salve, chewing gum, pastilles, sachets, mouthwashes, tablets, including effervescent tablets, dental floss, plasters, bandages, sheets, foams, films, sponges, dressings, drenches, bioabsorbable patches, sticks, and the like.

Also claimed herein are methods of administering the disclosed compositions to treat or prevent/delay progression of periodontal pathologies in mammals, preferably humans. Also contemplated are veterinary uses for the compositions, particularly administration to dogs, pets, and farm animals. The compositions of the present invention are effective and provide continuous protection when administered to a patient between 1 and 4 times a day.

III. Treatment and Prevention of Pathologies Around Medical Implants and Dental Implants

In one aspect, the invention provides a method of treating or preventing progression of pathology around a medical implant or dental implant. For example, a method of treating or preventing/delaying progression of inflammation around an implant.

The use of dental implants has revolutionized the treatment of partially and fully edentulous patients today. While in many cases dental implants have been reported to achieve long-term success, they are not immune from complications associated with peri-implant mucositis and peri-implantitis; inflammatory conditions in the soft and hard tissues at dental implants.

Peri-implant mucositis has been described as a disease in which the presence of inflammation is confined to the soft tissues surrounding a dental implant with no signs of loss of supporting bone following initial bone remodeling during healing. Peri-implantitis has been characterized by an inflammatory process around an implant, which includes both soft tissue inflammation and progressive loss of supporting bone beyond biological bone. The description of the inflammatory process of peri-implant mucositis around an implant is quite similar to gingivitis around natural teeth. Shortly after implants are placed, glycoproteins from saliva adhere to exposed titanium surfaces with concomitant microbiological colonization. The formation of a biofilm plays a significant role in the initiation and progression of peri-implant diseases and is essential for the development of infections around dental implants. Moreover, peri-implant diseases have been associated with Gram-negative anaerobic bacteria similar to those found around natural teeth in patients with severe chronic periodontitis.

It is generally accepted that peri-implant mucositis is the precursor of peri-implantitis, as it is accepted that gingivitis is the precursor of periodontitis. However, similar to the causal relationship between gingivitis and periodontitis, peri-implant mucositis does not necessarily progress to peri-implantitis. The “epithelial sealing” around implants is similar in function to that around teeth. Moreover, it is concluded that there is no evidence to suggest that any structural differences between natural teeth and implants would significantly alter the host response to bacterial challenge. Furthermore, there is evidence to suggest that peri-implant mucositis, like gingivitis, is reversible when effectively treated. Thus, elimination of the biofilm from the implant surface is the prime objective when treating peri-implant mucositis. Peri-implantitis, like periodontitis, occurs primarily as a result of an overwhelming bacterial insult and subsequent host immune response.

Outcomes from animal and human cross-sectional studies have found that the bacterial species associated with periodontitis and peri-implantitis are similar, mainly Gram-negative aerobes. Moreover, Staphylococcus aureus may also be an important pathogen in the initiation of peri-implantitis. Studies have shown that peri-implantitis and periodontitis lesions from human biopsies have many features in common. The connective tissue adjacent to the pocket epithelium is infiltrated by inflammatory cells, with B-lymphocytes and plasma cells being the most dominating cell types. Basically, similar markers are upregulated between peri-implantitis and periodontitis, including proinflammatory cytokines such as interleukin (IL)-1β, IL-6, IL-8, IL-12, and tumor necrosis factor (TNF)-alpha. Although sharing similarities with periodontitis in both the bacterial initiators and key immune components to those insults, the rate of disease progression and the severity of inflammatory signs for peri-implantitis may be similar. Experiments that allowed undisturbed dental plaque formation on implants and teeth in humans and in dogs demonstrated more advanced inflammatory cell infiltration in the peri-implant mucosa. Features of experimentally created peri-implantitis and periodontitis have been compared. The results suggested that clinical and radiographic signs of tissue destruction were more pronounced and the size of inflammatory cell infiltrate in the connective tissue was larger, approaching the crestal bone in peri-implantitis. The increased susceptibility for bone loss around implants may be related to the absence of inserting collagen fibers into the implant as is the case with a tooth.

A recent comparison of periodontitis and peri-implantitis noted a “self-limiting” process existing in the tissues around natural teeth that resulted in a protective connective tissue capsule of the supracrestal gingival fibers of the tooth that separated the lesion from the alveolar bone. Another distinct feature in studies on experimentally induced peri-implantitis was that following ligature removal, there was spontaneous continuous progression of the disease with additional bone loss. All implants appear to be susceptible to peri-implantitis. Hence, the primary objective for treating peri-implantitis is similar to that for treating peri-implant mucositis, which is the elimination of the biofilm from the implant surface. Dr. Paul Rosen, chair; Drs. Donald Clem, David Cochran, Stuart Froum, Bradley McAllister, Stefan Renvert, Hom-Lay Wang. Peri-Implant Mucositis and Peri-Implantitis: A Current Understanding of Their Diagnoses and Clinical Implications. This paper was developed under the direction of the Task Force on Peri-Implantitis and approved by the Board of Trustees of the American Academy of Periodontology in January (2013).

Medical implants often fail as a result of so-called foreign body reactions during which inflammatory cells are recruited to implant surfaces. Despite the clinical importance of this phenomenon, the mechanisms involved in these reactions to biomedical implants in humans are not well understood. The results from animal studies suggest that both fibrinogen adsorption to the implant surface and histamine release by local mast cells are involved in biomaterial-mediated acute inflammatory responses. Plasma coated implants accumulated significantly more phagocytes than did serum coated implants and the recruited cells were predominantly macrophage/monocytes. Administration of both H1 and H2 histamine receptor antagonists greatly reduced the recruitment of macrophages/monocytes and neutrophils on implant surfaces. In humans—as in rodents—biomaterial-mediated inflammatory responses involve at least two crucial events: histamine-mediated phagocyte recruitment and phagocyte accumulation on implant surfaces engendered by spontaneously adsorbed host fibrinogen. Based on these results, we conclude that reducing fibrinogen-surface interactions should enhance biocompatibility and that administration of histamine receptor antagonists prior to, and shortly after, medical device implantation should improve the functionality and longevity of medical implants.

Although most implant materials are inert, non-immunogenic and non-toxic, devices made of such materials still often trigger a variety of adverse reactions. These include surface-mediated thrombosis associated with blood contact surfaces, complement activation induced by hemodialysis membranes, inflammation surrounding many types of implants, device-centered infections and fibrotic tissue formation around tissue implants and prostheses. These complications may cause the failure of many types of medical implants, often requiring surgical removal and replacement, increasing both the risk to patients and the cost of health care. Consequently, intensive research efforts have been devoted to the development of novel strategies to improve tissue compatibility of medical devices. However, improvements in biocompatibility have been hindered by our lack of understanding of the basic mechanisms involved in human tissue responses to biomaterial implants. Because biomaterials spontaneously accumulate a layer of adsorbed plasma proteins prior to inflammatory cell accumulation, it is widely accepted that the types and species of adsorbed proteins play an important role in the pathogenesis of biomaterial-mediated acute inflammatory responses. Using an animal implantation model, it has been previously found that spontaneously adsorbed (and partially denatured) fibrinogen is a critical mediator of acute inflammatory responses to biomaterial implants. In line with this, much less phagocyte accumulation was observed on the surfaces of serum-coated implants than on implants coated with plasma.

The composition described herein can be used as a coating for a dental implant. Such coating may be applied during the manufacturing process, or applied at bedside before the implant is placed in a patient. Alternatively, the composition may be used as a pre-implantation rinse. One can wash the dental implant in a solution prior to implantation.

As disclosed and exemplified herein, the compositions of the invention can be used, for example, for the treatment and/or prevention (including delaying the progression) of inflammatory diseases, in particular around medical implantable devices and dental implants, as well as oral infectious disease, especially mucositis and peri-implantitis.

A skilled clinician can determine a suitable safe and effective dose empirically. Typically, the compositions described herein can be administered as needed to achieve the desired effect, sometimes up to about 3 or 4 times a day. Methods of determining the most effective method and dosages of administration are well known to those of skill in the art and will vary with the pharmaceutical composition, the severity of the disease, and the subject being treated. For example, an effective dose can be a dose that delay or prevent the progression gingivitis to periodontitis or peri-implantitis, to ameliorate the worsening of gingivitis, or to reduce the size of periodontal pocket, to restore the barrier function of oral gingival tissues, or to restore electrical charge (e.g., TEER) of tight junction.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Preparation of Mucoadhesive Compositions

Provided below are exemplary mucoadhesive formulations. Percentages are expressed in parts by weight.

### 1. Mouth-Wash

| | |
|---|---|
| Cimetidine | 0.50% |
| N,O-Carboxymethyl chitosan | 0.50% |
| 70% Sorbitol FU | 47.67% |
| Sodium benzoate | 1.00% |
| Xylitol | 0.60% |
| Disodium EDTA | 0.013% |
| Flavors | 0.30% |
| Dye E 124 | q.s. |
| Citric acid | q.s. to pH 6.8-7.2 |
| Osmosized water | q.s. to 100% |

### 2. Oral Gel

| | |
|---|---|
| Cimetidine | 0.60% |
| N,O-Carboxymethyl chitosan | 0.70% |
| Sodium alginate | 1.20% |
| 70% Sorbitol FU | 5.00% |
| Preservative | q.s. |
| Flavors | q.s. |
| Dye E 124 | q.s. |
| Osmosized water | q.s. to 100% |

### 3. Oral Spray

| | |
|---|---|
| Cimetidine | 0.60% |
| N,O-Carboxymethyl chitosan | 0.40% |
| Allantoin | 0.20% |
| Preservative | q.s. |
| Flavors | q.s. |
| Osmosized water | q.s. to 100% |

### Example 2

### Characterization of Mucoadhesive Compositions

1. Oral Tissue Model

Tissue-engineered oral mucosal models have been developed for clinical applications and also for in-vitro studies of biocompatibility, mucosal irritation, disease, and other oral biology phenomena. The development of tissue-engineered models of oral disease has enhanced the understanding of disease progression and simplified the study of therapeutics. Dongari-Battzoglou, A., et al. Development of a highly reproducible three-dimensional organotypic model of the oral mucosa. Nat Protoc. 2006; 1(4): 2012-2018; Moharamzadeh, K., et al., Tissue-Engineered Oral Mucosa; a Review of Scientific Literature, J Dent Res 86 (2):115-124, 2007; Schmalz G., et al. Release of prostaglandin 2, IL6 and IL-8 from human oral epithelium culture models after exposure to compounds of dental materials. Eir. J. Oral. Sci., 108:442-448 (2000).

The regulatory framework in Europe strongly suggests moving from animal based pre-clinical studies to an in vitro approach based on alternatives. Relevant in the pharmaceutical industry is the Directive 2010/63/UE that in the Article 47, states:
“The Commission and the Member States shall contribute to the development and validation of alternative approaches which could provide the same or higher levels of information as those obtained in procedures using animals, but which do not involve the use of animals or use fewer animals or which entail less painful procedures, and they shall take such other steps as they consider appropriate to encourage research in this field.”

The pharmaceutical industry is currently working to implement biologically relevant models to perform product testing in a more ethical and scientific framework the assessment of topical products (including medical devices) with the final aim to use a more accurate and sensitive tool to evaluate efficacy and safety.

In vitro 3D reconstructed human living tissue models issued from the airlift technique are recognized as being a sensitive and reliable model for in vitro skin, eye and mucosae compatibility testing in order to replace animals (ATLA 33 Suppl. 1, 47-81. 2005) in the hazard identification and safety evaluation of topically applied products. From a regulatory point of view several 3D human tissue models are the biological model used as Replacement Alternatives for the hazard classification of chemicals, ex. for skin irritation potential (OECD 439) or for skin corrosivity (OECD 431). The advantage of using human 3D tissue models is their higher sensitivity compared to human tissue resulting in better discrimination between products, higher reproducibility of the results and compared to cell-monolayers, they give the possibility to test the product in the same doses and mode of use as in vivo on humans. Thus, experimental results in such models are highly probative that tested compositions will function in vivo in the same manner.

The experimental approach adopted by VitroScreen is the Multiple Endpoints Analysis (MEA, see Meloni et al., Occludin gene expression as an early in vitro sign for mild eye irritation assessment, Toxicol In Vitro. 2010 February; 24(1):276-85. doi: 10.1016/j.tiv.2009.08.016. Epub 2009 Sep. 1; Available at http://www.vitroscreen.com/ardocCM/fput.php/3d26cc38eb62027f765c12eaecb16eeae6fd2133097f93ae3e8daa840a07d51078093cb0694e4c3309dd950c9c7c9f6c65388a4ce7ab86e7255640c70f6624210592f66a597032b7e20613a79da18flb/2002%20MULTIPLE%20ENDPOINTS%20ANALYSIS.pdf) that allows a global assessment of product interaction with the living tissue by the investigation of complementary and relevant cellular, biochemical, physical and morphological parameters after single acute exposure or long term tolerance if applicable.

A commercially available in vitro model of oral epithelium, developed by SkinEthic Laboratories, consists of a three dimensional, multilayer culture of the TRI46 keratinocyte cell line on polycarbonate cell culture inserts. This tissue model forms a non-keratininzing oral epithelium that has been extensively used for biocompatibility studies and other clinical applications. The model is a three dimensional reconstructed human oral and gingival epithelium that forms multilayer, stratified non-keratinized and keratinized oral epithelia, respectively. The model tissues exhibit in vivo-like morphological and growth characteristics. Both reconstructed tissues express cytokeratin K13 and weakly express cytokeratin K14. They also produce naturally occurring antimicrobial peptides, including human beta defensins.

Tissue cultures are recognized as being a sensitive and reliable model for in vitro skin, eye and mucosae compatibility testing in order to replace animals (7^{th} Amendment of EEC 76/768) and to improve the prediction of irritants (ATLA 33 Suppl. 1, 47-81. 2005) in the safety evaluation of topically applied products.

2. Purpose

An experimental model based on 3D human in vitro reconstructed oral mucosal tissue was used to assess the membrane barrier, film forming and protective properties of a series of 6 new formulations to be registered as Medical Device Class III comprising a combination of cimetidine and N,O-carboxymethyl-chitosan. A placebo was also tested. The goals of the study were to:
identify the optimal exposure time for film forming,
better understand the contribution of each component of the cimetidine combination to the film forming properties
obtain information about the compatibility of the formulations with the oral mucosa.

The following assays were performed on each sample:
TEER, which reflects the global resistance of the epithelial barrier linked to the integrity of tight junction structure and to epidermal thickness. An increase in TEER without thickness modification suggests a film forming property and protective efficacy. A decreased TEER value indicates a reduced fence property measured at the tight junction level linked to intracellular or extracellular modifications. Stable TEER values (no modifications) are linked to a neutral effect on barrier function and fence properties.
Lucifer Yellow paracellular passage is an assay used to verify the integrity of cell junctions and tissue permeability modifications after treatment. When the tight junctions are unbroken, Lucifer Yellow has a very low permeability. If the junctions are damaged, Lucifer Yellow flow is much greater.
LDH release allows a determination of membrane integrity by measuring lactate dehydrogenase (LDH) in the extracellular medium. This enzyme is normally present in the cytosol, and cannot be measured extracellularly unless cell damage has occurred

3. Experimental Design

The protocol was carried out on duplicate tissues. Formulation No. 3 (0.5% cimetidine+0.5% N,O-carboxymethyl-chitosan) was applied to the epithelial surface (500 μL) for 5, 10, or 20 minutes. After washing the cells to remove the formulation, TEER was measured. The results, depicted in FIG. 2, showed a slight increase in TEER values after 10 minutes of exposure linked to film forming properties that were not confirmed after 20 min where the TEER was reduced compared to basal values indicating a possible penetration of the product. According to the results obtained, the exposure time of 10 minutes was selected for the study performed with all 6 formulations because the increase in TEER at this stage indicated that the composition had formed a film over the tissue. TEER was measured for each epithelial tissue sample. Each product was applied on the epithelial surface (500 μL). The cells were exposed to the formulation for ten minutes, and then washed. TEER, LDH and Lucifer Yellow paracellular flux were then assessed. An increase in TEER corresponds to a film forming property, while a reduction in TEER is linked to the penetration of the formulation. Moreover, one tissue sample exposed to each formulation was fixed in formalin for further histological investigation.

4. Materials

Tissue Model

The SkinEthic© Reconstituted Human Oral Epithelium (RHO) of 0.5 cm² was used for the evaluation. Transformed human keratinocytes (Cutaneous carcinoma derived cell line, TR146) were deposed on an inert polycarbonate filter and cultured at the air-liquid interface for 5 days in a chemically defined medium in order to form a structured epithelium.

The tissue and media were manufactured in compliance with ISO 9001. The intended use of the biological model is for research purposes only (standardized in vitro testing of chemicals or formulations).

Each RHO batch has been tested for the absence of HIV, Hepatitis B, Hepatitis C, Mycoplasma. The inserts containing the RHO at day 5 were shipped at room temperature in a multiwell plate filled with an agarose nutrient solution in which they were embedded. The maintenance medium has been prepared by the manufacturer under aseptic conditions. The plates were sealed with a white tape and packed sterile in an aluminum bag. The expiration date of tissue and media was indicated in the technical data and safety sheet of each batch. Media was stored at 2-8° C. protected from light.

Immediately after the arrival in the laboratory the RHO were removed from the agarose nutrient solution under a sterile air flow cabin. The inserts were rapidly placed in a 12-well plate previously filled with 0.75 mL of the SkinEthic maintenance medium at room temperature. The wells were placed in an incubator at 37° C., 5% CO2 and saturated humidity overnight. The test was started the day after the arrival.

The negative control was saline solution (0.9% NaCl) which is characterized by its neutral action on tissues.

| | NEGATIVE CONTROL |
|---|---|
| NAME | Saline solution 0.9% |
| UNIVOCAL CODE | NC |
| BATCH/ | G-036/3 |
| MANUFACTURER | EUROSPITAL |
| STORAGE | 15° C.-25° C. |
| DOSE | 500 μL |
| EXPIRATION DATE | 08.2015 |
| MSDS | YES |
| CERTIFICATE OF ANALYSIS | YES |

Test Compositions

| | NAME | | | | | |
|---|---|---|---|---|---|---|
| | | | Cimetidine | Cimetidine | Cimetidine | |
| | Placebo | | 0.5% + N,O- | 0.5% + N,O- | 0.5 + N,O- | N,O- |
| | (Excipients used | Cimetidine | carboxymethyl- | carboxymethyl- | carboxymethyl- | carboxymethyl- |
| | in example 5) | 0.5% | chitosan 0.5% | chitosan 0.1% | chitosan 0.4% | chitosan 0.5% |
| Formulation No. | 1 | 2 | 3 | 4 | 5 | 6 |
| pH | 6.73 | 7.22 | 7.25 | 7.24 | 7.21 | 6.94 |
| DOSE | 500 μL | 500 μL | 500 μL | 500 μL | 500 μL | 500 μL |
| STORAGE | Room tem- | Room tem- | Room tem- | Room tem- | Room tem- | Room tem- |
| | perature | perature | perature | perature | perature | perature |

Reagents

| REAGENT | MANUFACTURER | BATCH |
|---|---|---|
| Maintenance Medium | SKINETHIC | 12 011J M040 |
| Saline Solution | EUROSPITAL | G-036/3 |
| Cytotoxicity Detection Kit-LDH | ROCHE | 13651900 |
| Lucifer Yellow | SIGMA-ALDRICH | MKBHO593V |

5. Methods

TEER

TEER was measured for each sample. 0.5 mL of saline solution was directly applied onto RHO tissue in a 6 well plate containing 5 mL of saline solution. The instrument Millicell-ERS (range 0-20 kΩ) was placed with the two electrodes in the two chambers: the measure directly appeared on the display and it was reported in the laboratory notebook.

Three measurements for each tissue were taken. Due to variability within the tissues the measurement done at t=0 was taken as a basal value and reference for each tissue sample. The blank value (insert without tissue) was subtracted to the sample value (mean 3 measurements). This result was then corrected considering the tissue surface (0.5 cm²).

Ω (mean 3 measurements) sample−Ω blank=Ω×tissue surface (0.5 cm²)

LDH

The cell membrane forms a functional barrier around the cell, and traffic into and out of the cell is highly regulated by transporters, receptors and secretion pathways. When cells are damaged, they become ‘leaky’ and this forms the basis for the second type of assay. Membrane integrity is determined by measuring lactate dehydrogenase (LDH) in the extracellular medium. This enzyme is normally present in the cytosol, and cannot be measured extracellularly unless cell damage has occurred.

A commercially available kit (Cytotoxicity Detection KIT-LDH, Roche) has been used to quantify the LDH released in culture media by a colorimetric assay based on formazan salt detection 492 nm with reference at 690 nm). The culture supernatant is collected and incubated with the reaction mixture included the kit (20 min, room T°, in the dark). An increase in the amount of dead or plasma membrane-damaged cells results in an increase of the LDH enzyme activity in the culture media. This increase in the amount of enzyme activity in the supernatant directly correlates to the amount of formazan formed during a defined time period therefore, the amount of color formed in the assay is proportional to the number of lysed cells.

A standard curve using different concentrations of LDH: 125; 62.5; 31.25; 15.63; 7.81; 3.91; 1.95 mU/mL has been previously determined

Lucifer Yellow

Lucifer yellow (“LY”) is a fluorescent dye impermeable to the cell membrane. It is used to study the paracellular permeability of a substance. When the junctions are unbroken, Lucifer Yellow has a very low permeability; if the joints are damaged, Lucifer Yellow flow will be much greater. Therefore this assay is used to verify the integrity of cell junctions in the presence of the substance to be evaluated.

0.5 mL of Lucifer Yellow (500 μM in saline solution) was applied in the apical compartment (into the RHO insert) after exposure to the substance to be tested. 1 mL of saline solution was added in the basolateral compartment. The transport of LY was assessed as a switch from apical to basolateral compartment after the defined incubation period of 30 minutes at 37° C. previously determined.

The reading was performed in the spectrofluorimeter (TECAN INFINITE M200) with 428 nm excitation and 535 nm emission. The measurement of fluorescence (RFU) is done at apical and basolateral level and flux was calculated with the following formula (bl=basolateral; ap=apical):

LY Flux %=(RFU BL/RFU APₜ₌₀)×100

6. Results

Preliminary Test

An assessment of the impact of the formulation type on the fence properties of RHO has been performed. The product was applied and then washed off before the TEER measurement. The results showed a slight increase of TEER values after 10 minutes of exposure. This time frame represents a realistic exposure (permanence on the mouth mucosa) for this type of product and it has been adopted for the screening of the formulations.

TEER Measurement after 10 Min Exposure

The TEER values before treatment (T=0 h) were in the accepted range (70-85 OHM*cm²) according to internal data and they reflect the global resistance of the barrier linked both to the integrity of tight junction structure and to the epithelial thickness.

None of the products significantly reduced the TEER. This corresponds to no modification of the electrical resistance associated with the integrity of the tight junctions. None of the products disturbed tissue homeostasis and had no negative impact (i.e., reduction) of the paracellular flux.

Product No. 4 has determined the highest increase in TEER, showing a film forming property (+7%).

LDH Release

Lactate dehyrogenase (LDH) release in the culture medium was measured to quantify the membrane integrity. This enzyme is normally present in the cytosol, and cannot be measured extracellularly unless cell damage has occurred.

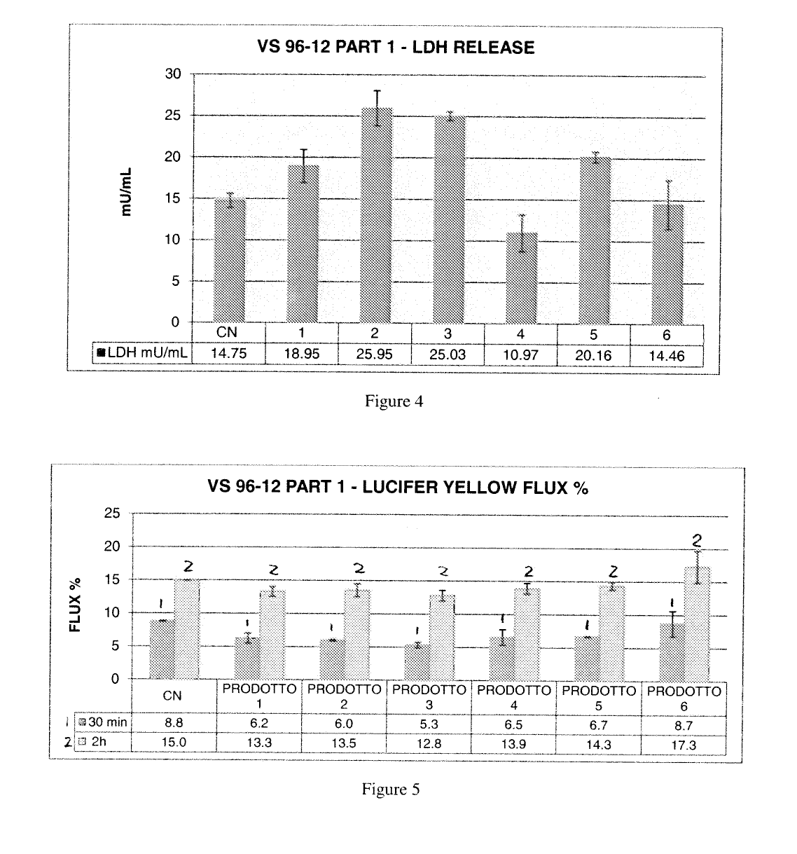

As shown in FIG. 4 the released LDH was under the significant values for barrier impairment detection and furthermore not different form the negative control values.

Product 4 reduced the LDH release to nearly undetectable values (10 mU/mL), suggesting a protective mechanism at the barrier level. However all of the values generated are close to the method detectable limits and are not significantly different. This confirms the compatibility of the formulations with the mucosal living tissue.

LY Assay

Lucifer yellow assay measures the modification of RHO permeability by using a fluorescent probe as marker of paracellular flux. Fluorescence results are reported as flux %. The LY assay is a very sensitive measure of the tight junction structure and integrity: when the tight junctions are unbroken, Lucifer Yellow has a very low permeability; if these joints are damaged, Lucifer Yellow flow is much higher.

Taking into account the measure done after 30 min application of LY all the formulations, included the placebo (1) have reduced the paracellular flux=less permeable tissue, as shown in FIG. 5.

The most effective formulation was formulation No. 3, and the least effective is formulation No. 6.

Conclusion

The results of the study are summarized in Table 1:

TABLE 1
Summary of Example 2 test results.

| | | RESULTS compared to UNTREATED | | |
|---|---|---|---|---|
| | | CONTROL tissue | | |
| | | | | LUCIFER |
| | | | | YELLOW |
| | | | | FLUX |
| | | | | as recorded |
| | | | | at 30 minutes |
| | | TEER | | Product |
| | | 10 min | LDH | treatment: |
| PRODUCT | No. | treatment | RELEASE | 10 minutes |
| Placebo | 1 | slight | not | reduction |
| | | reduction | different | |
| Cimetidine 0.5% | 2 | not | not | reduction |
| | | modified | different | |
| Cimetidine 0.5% + N,0- | 3 | not | not | reduction |
| N,O-carboxymethyl- | | modified | different | |
| chitosan 0.5% | | | | |
| Cimetidine 0.5% + N,0- | 4 | 7% | reduction | reduction |
| N,O-carboxymethyl- | | increase | | |
| chitosan 0.1% | | | | |
| Cimetidine 0.5 + N,0- | 5 | slight | not | reduction |
| N,O-carboxymethyl- | | reduction | different | |
| chitosan 0.4% | | | | |
| N,O-carboxymethyl- | 6 | not | not | not different/ |
| chitosan 0.5% | | modified | different | slightly |
| | | | | increase |

The placebo (Product 1) has slightly reduced the TEER and seems not to be able to form a film on the epithelial surface; furthermore it has reduced by itself the paracellular flux >from Control value 8.8 to 6.2)

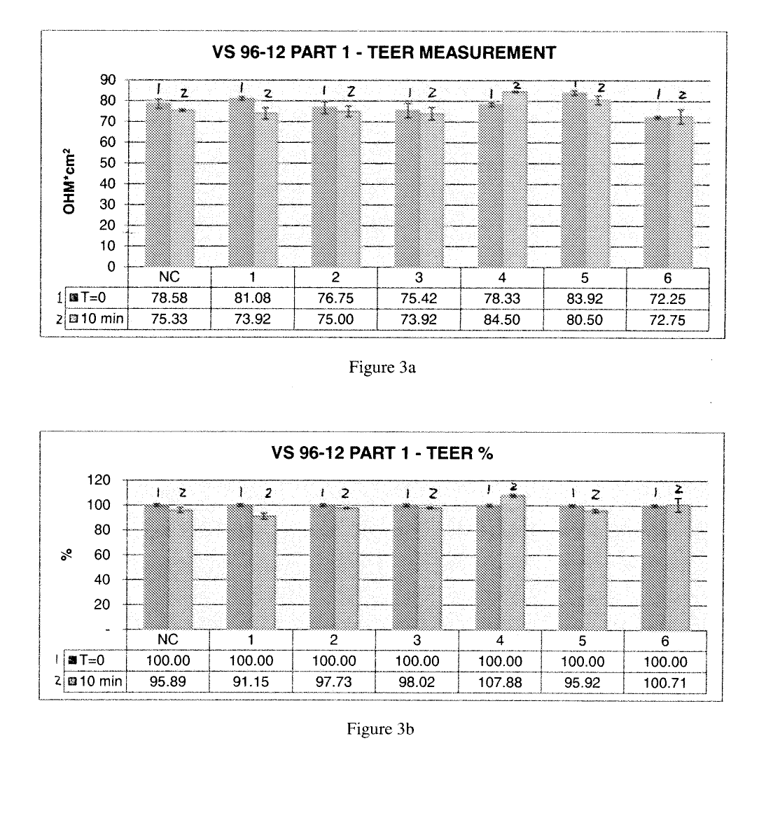

Cimetidine alone does not have mucoadhesive property. Product 2 or cimetidine alone did not show any immediate protection, or any significant effect on permeability, and did not have a direct mucoadhesive property as demonstrated in the TEER measurement (FIG. 3a). TEER, assessed by both absolute and normalized values, was not different from the control and a reduction of the LY paracellular flux was observed (from Control value 8.8 to 6). The efficacy of the cimetidine in modifying the permeability has been demonstrated by comparing the results of product 2 (cimetidine alone at 0.5%) to product 6 (N,O-carboxymethyl-chitosan alone 0.5%). The cimetidine was able to counteract the N,O-carboxymethyl-chitosan direct action by reducing the paracellular flux and enhancing the penetration. Product 2 showed protective action by maintaining the physiological permeability of the mucosa when it is modified or combined with another compound; in this case the penetration enhancer N,O-carboxymethyl-chitosan

None of the products caused damage to the tissues because the levels of LDH released were not significantly different from those measured in the untreated control and were close to the detectable limit.

Product Nos. 3 and 4 have both determined an epithelial flux reduction suggesting better epithelial fence properties and a tissue protection against barrier damage according to LDH release results.

It is possible to conclude that there was film forming activity according to the increase of TEER values. In this experiment, Product 4 has shown better film forming activity in non-injured tissues.

Formulation No. 4 represents a positive synergy compared to the formulation No. 2 based on the interesting increase of TEER values; a positive influence on the epithelial permeability (reduced LY values) has been also demonstrated compared to Control tissue and with similar results compared to Products 1-2-and 5.

Formulation No. 3 represents a positive synergy as far as permeability flux results are considered (LY) and it shows the highest efficacy in reducing the permeability (from 5.3 compared to 8 of the Control).

Cimetidine alone did not have direct mucoadhesive properties and did not show immediate protection as demonstrated by the TEER measurement, as shown in FIG. 3a. The testing of cimetidine/N,O-carboxymethyl-chitosan products has shown that adding combining the two product adds a mucoadhesive film forming property to cimetidine. The composition has demonstrated membrane barrier and protective action, maintaining the integrity associated with the tight junctions without modifying the electrical resistance, as exemplified with Formulation Nos. 3 and 4.

### Example 3

### Film Forming, Tissue Restoring and Protective Efficacy of the Mucoadhesive Formulations

1. Purpose

An experimental model based on a mechanically injured 3D human in vitro tissue of oral mucosa was proposed to assess a membrane barrier function through film forming, tissue restoring and protective efficacy of new formulations to be registered as Medical Devices Class III containing a complex of cimetidine-N,O-carboxymethyl-chitosan.

The test items have been tested compared to positive and negative controls after a 10 minute exposure to each control and test composition (exposure time selected according to the preliminary results, described previously, on injured tissues followed by a 1 h and 6 h recovery period. TEER, LY, and LDH were evaluated.

2. Methods

The protocol has been carried out on duplicate tissues for the following series of test situations:
Negative control: not injured—RHO treated with saline solution
Positive control: Injured RHO control
Not injured but treated RHO sample (to conclude about film forming in absence of injury)
Injured and treated RHO

The formulation tested were Cimetidine 0.5%+N,O-carboxymethyl-chitosan 0.5%, identified with code No. 3, and Cimetidine 0.5%+N,O-carboxymethyl-chitosan 0.1%, identified with code No. 4.

At t=0 the cells were measured for TEER and then subsequently mechanical injured with a brush. 500 μL of the undiluted product or negative control (Saline solution on tissue not injured) were directly applied on the oral epithelium for 10 minutes at room temperature. As a positive control an injured and untreated tissue sample was also included.

After 10 min, the solutions were removed by suction, without washing, and tissues were been incubated for 1 h and 6 h in order to assess the recovery of the injury over time.

In order to follow the epithelial barrier recovery, TEER measurement has been performed before treatment (t=0 basal value), immediately after injury and after 1 h and 6 h recovery.

Lucifer yellow paracellular flux was assessed after 10 min, 1 h and 6 h recovery in duplicate and one tissue per series was collected for further histological analysis.

At the end of each exposure, according to the specific protocol, the LDH release assay was been performed on media.

3. Materials

The SkinEthic© Reconstituted Human Oral Epithelium of 0.5 cm² was used for the evaluation. Transformed human keratinocytes (Cutaneous carcinoma derived cell line, TR146) were deposed on a inert polycarbonate filter and cultured at the air-liquid interface for 5 days in a chemically defined medium in order to form a structured epithelium. The tissue and media were manufactured in compliance with ISO 9001. The intended use of the biological model is for research purpose only (standardized in vitro testing of chemicals or formulations).

Each RHO batch has been tested for the absence of HIV, Hepatitis B, Hepatitis C, Mycoplasma. The expiration date of tissue was indicated in the technical data and safety sheet of each batch. Media was stored at 2-8° C. protected from light.

| | | |
|---|---|---|
| NAME and BATCH | RHO/S/5 | STATE |
| | 13 022B 0102 | |
| MANUFACTURER | SKINETHIC | |
| | LABORATORIES | |
| THICKNESS | 80 μm | |
| HISTOLOGY AT DAY 5 | 6 | Accepted |
| (layers ≧4) | | |
| CELL VIABILITY AT | 1.151 ± 0.052 | Accepted |
| DAY 5 | | |
| (O.D. ≧0.8) | | |
| DATE OF ARRIVAL | 15.01.2013 | |
| EXPIRATION DATE | 21.01.2013 | |
| WAYBILL No. | DHL 80 3241 2426 | |

Immediately after the arrival in the laboratory the RHO were removed from the agarose nutrient solution under a sterile air flow cabin. The inserts were rapidly placed in a 12-well plate previously filled with 0.75 mL of the SkinEthic maintenance medium at room temperature. The wells were placed in an incubator at 37° C., 5% CO2 and saturated humidity overnight. The test was started the day after the arrival.

Test Compositions

| | | |
|---|---|---|
| NAME | Cimetidine 0.5% + N,O- | Cimetidine 0.5% + N,O- |
| | carboxymethyl- | carboxymethyl- |
| | chitosan 0.5% | chitosan 0.1% |
| Formulation No. | 3 | 4 |
| pH | 7.25 | 7.24 |
| DOSE | 500 μL | 500 μL |
| STORAGE | Room temperature | Room temperature |

Positive and Negative Controls.

| | | POSITIVE |
|---|---|---|
| | NEGATIVE CONTROL | CONTROL |
| NAME | Saline solution 0.9% | Injured |
| UNIVOCAL CODE | NC | INJ |
| BATCH/ | G179/3 | |
| MANUFACTURER | EUROSPITAL | |
| STORAGE | 15° C.-25° C. | |
| CONCENTRATION OF USE | As such | |
| DOSE | 500 μL | |
| EXPIRATION DATE | 09.2015 | |
| MSDS | NO | |
| CERTIFICATE OF | NO | |
| ANALYSIS | | |

NEGATIVE CONTROL was the saline solution (0.9% NaCl) which is characterized by its neutral action on tissues.
POSITIVE CONTROL for film forming and restoring efficacy was the injured tissue sample.

Instruments

| INSTRUMENT | MANUFACTURER |
|---|---|
| ANALYTICAL BALANCE XS 204 | METTLER-TOLEDO |
| pH-METER SEVEN MULTI | METTLER-TOLEDO |
| CO₂ INCUBATOR HERACELL NUAIRE | NUAIRE |
| LAMINAR FLOW CABIN NU 438-400 E | NUAIRE |
| SPECTROPHOTOMETER INFINITE M200 | TECAN |
| MILLICELL ERS | MILLIPORE |

Reagents

| REAGENT | MANUFACTURER | BATCH |
|---|---|---|
| MAINTENANCE MEDIUM | SKINETHIC | 13 011J M002 |
| SALINE SOLUTION | EUROSPITAL | G179/3 |
| Cytotoxicity Detection KIT-LDH | ROCHE | 13651900 |
| LUCIFER YELLOW | SIGMA-ALDRICH | MKBHO593V |

4. Methods

VitroScreen is a GLP certified laboratory for in vitro toxicology and it has adopted the GLP as unique quality system: according to this policy the study was conducted “according to the principles of GLP.”

TEER Measurement

The TEER is the measure of the movement of ions across the paracellular pathway regulated by polarized plasma membrane surfaces and by cell-to-cell tight junctions that together prevent movement of solutes and ions across the epithelia. TEER is an indirect assessment of tight junction stability and consequently is a direct measurement of the functionality of barrier function in epithelial tissue: it reflects the global resistance of the barrier linked both to the structure and to epithelial thickness.

Maintenance of stability and electrical resistance of an epithelium is critical for essential physiological processes, therefore significant changes in TEER may represent an early expression of cell damage and it can be considered a complementary parameter.

TEER was measured for each sample. 0.5 mL of saline solution was directly applied on the tissue placed in a 6 well plate containing 5 mL of saline solution as well. The instrument Millicell-ERS (range 0-20 kΩ) was placed with the two electrodes in the two chambers: the measure directly appeared on the display and it was reported in the laboratory notebook.

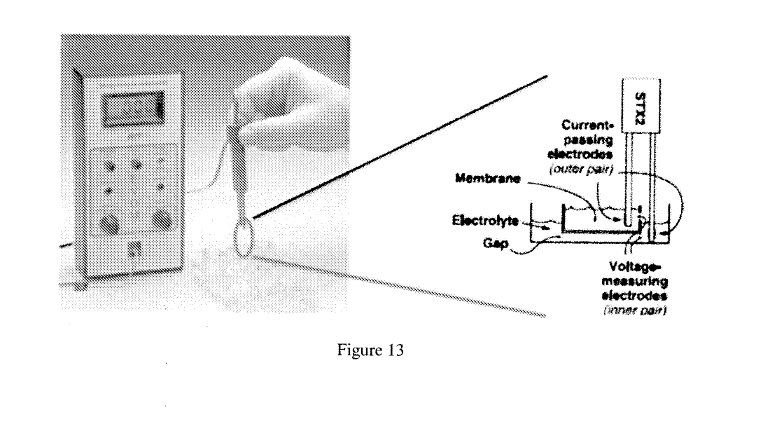

Three measurements for each tissue were done: because of variability within the tissues the measurement done at t=0 was taken as basal value and reference of each single tissue. The blank value (insert without tissue) was subtracted to the sample value (mean 3 measurements). This result was then corrected considering the tissue surface (0.5 cm²). See, FIG. 13.

Ω (mean 3 measurements) sample−Ω blank=Ω×tissue surface (0.5 cm²)

5. Millicell-ERS

LDH Release

The cell membrane forms a functional barrier around the cell, and traffic into and out of the cell is highly regulated by transporters, receptors and secretion pathways. When cells are damaged, they become ‘leaky’ and this forms the basis for the second type of assay. Membrane integrity is determined by measuring lactate dehydrogenase (LDH) in the extracellular medium. This enzyme is normally present in the cytosol, and cannot be measured extracellularly unless cell damage has occurred.

A commercially available kit (Cytotoxicity Detection KIT-LDH, Roche) has been used to quantify the LDH released in culture media by a colorimetric assay based on formazan salt detection 492 nm with reference at 690 nm). The culture supernatant is collected and incubated with the reaction mixture included the kit (20 min, room T°, in the dark). An increase in the amount of dead or plasma membrane-damaged cells results in an increase of the LDH enzyme activity in the culture media. This increase in the amount of enzyme activity in the supernatant directly correlates to the amount of formazan formed during a defined time period; therefore, the amount of color formed in the assay is proportional to the number of lysed cells.

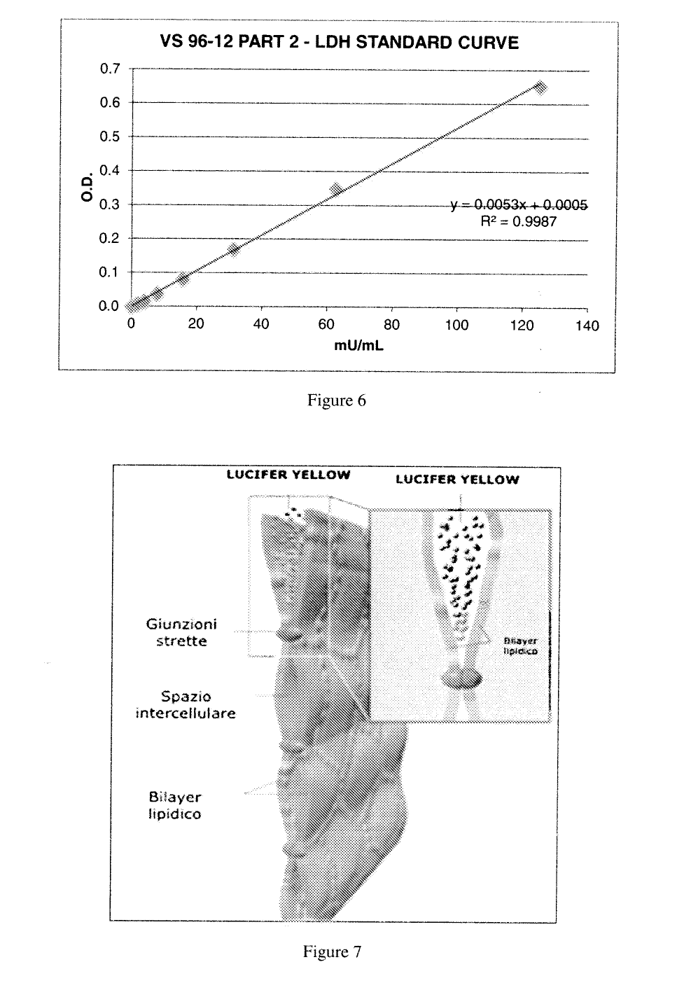

A standard curve using different concentrations of LDH: 125; 62.5; 31.25; 15.6; 7.8; 3.91; 1.95 mU/mL has been previously determined FIG. 6.

LY Assay

Lucifer yellow is a fluorescent dye impermeable to the cell membrane. It is used to study the paracellular permeability of a substance. When the junctions are unbroken, Lucifer Yellow has a very low permeability; if the joints are damaged, Lucifer Yellow flow will be much more higher. Therefore this assay is used to verify the integrity of cell junctions in the presence of the substance to be evaluated.

0.5 mL of Lucifer Yellow (500 μM in saline solution) were applied in the apical compartment (into the insert) after exposure to the substance to be tested. 1 mL of saline solution was added in the basolateral compartment. The transport of LY was assessed as a switch from apical to basolateral compartment after the defined incubation period of 30 minutes at 37° C. previously determined.

The reading was performed in the spectrofluorimeter (TECAN INFINITE M200) with 428 nm excitation and 535 nm emission. The measurement of fluorescence (RFU) is done at apical and basolateral level and flux was calculated with the following formula (bl=basolateral; ap=apical):

LY Flux %=(RFU BL/RFU APₜ₌₀)×100

6. Results of Statistical Analysis

TEER Measurement

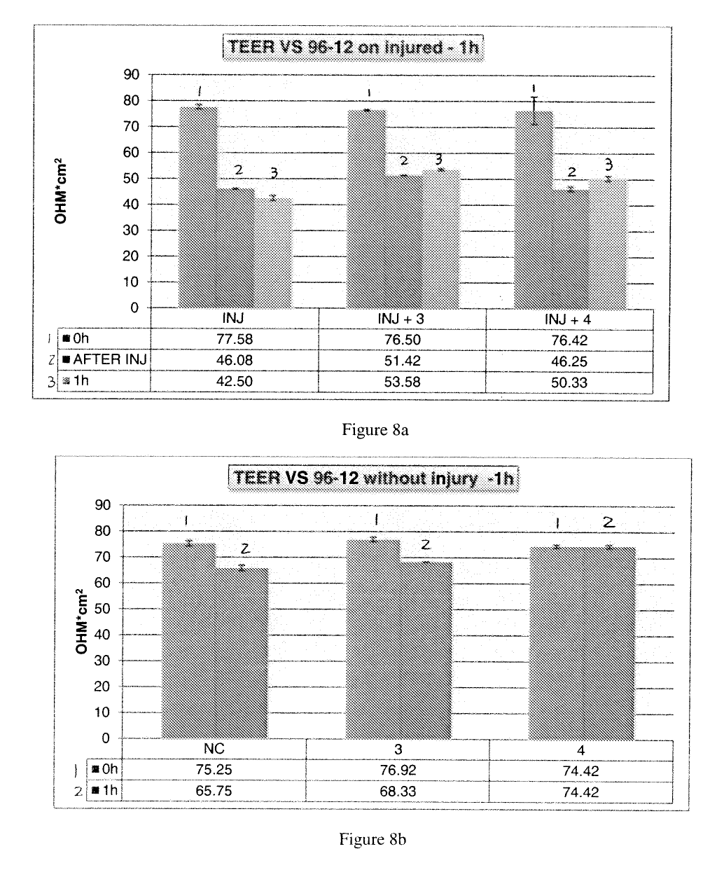

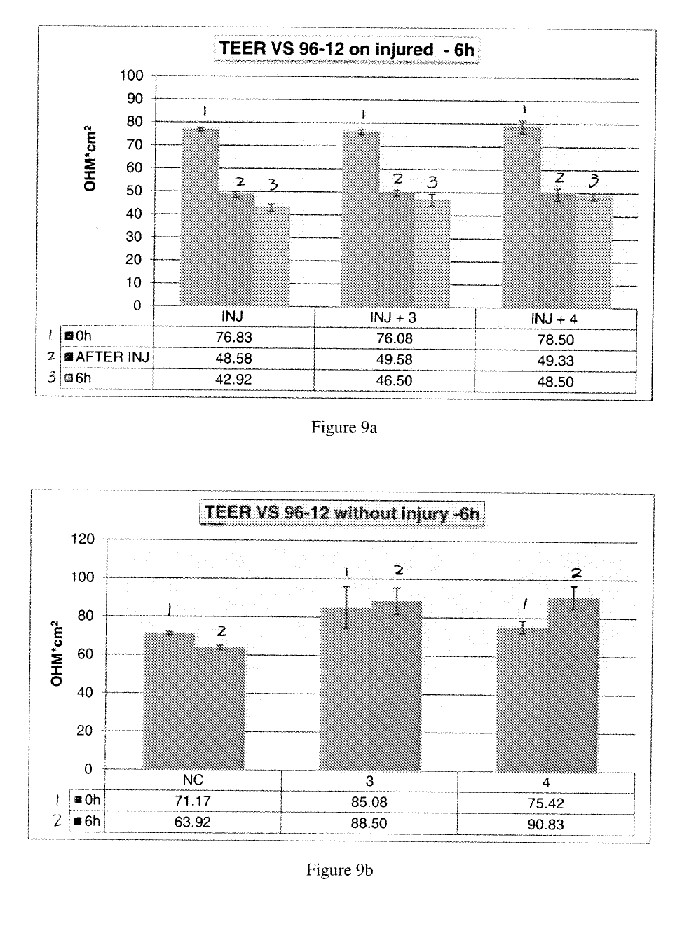

The TEER values of RHO before treatment (T=0 h) were in the normal, acceptable range (70-85 OHM*cm²). TEER results are reported in Table 2, Table 3, FIG. 8a, FIG. 8b, FIG. 9a and FIG. 9b.

TABLE 2
TEER measurement after 10 min + 1 h, as depicted in FIG. 8a

| | OHM * cm² ± SD | | |
|---|---|---|---|
| SAMPLE | 0 h | After injury | 1 h |
| NC | 75.25 ± 1.06 | / | 65.75 ± 1.06 |
| INJ | 77.58 ± 0.82 | 46.08 ± 0.12 | 42.50 ± 0.94 |
| 3 | 76.92 ± 0.82 | / | 68.33 ± 0.00 |
| INJ + 3 | 76.50 ± 0.24 | 51.42 ± 0.12 | 5358 ± 0.35 |
| 4 | 74.42 ± 0.59 | / | 74.42 ± 0.59 |
| INJ + 4 | 76.42 ± 5.30 | 46.25 ± 0.82 | 50.33 ± 0.94 |

After mechanical injury, as expected TEER values were strongly reduced in all injured samples (from about 77 OHM*cm² to 46.08-INJ), confirming internal data. It is important to underline that the values quantified after injury correspond to a reduced fence property measured at the tight junction level. The positive control injured tissue sample (INJ) showed a 7% reduction of TEER after 1 h post injury (from 46.08 to 42.50 OHM*cm²).

In injured tissues treated with formulations No. 3 and No. 4 the values measured immediately after 1 hour recovery are slightly higher compared to the control sample results: respectively+4% for formulation No. 3 and +9% for formulation No. 4.

FIG. 8b reports the TEER values measured on RHO treated with product No. 3 and No. 4 without injury. Both samples exhibited TEER values comparable to those found in the Negative Control sample. The negative control showed a 14% TEER reduction compared to stable value of product No. 4.

TABLE 3
TEER measurement after 10 min + 6 h, as depicted in FIG. 9a.

| | OHM * cm² ± SD | | |
|---|---|---|---|
| SAMPLE | 0 h | After injury | 6 h |
| NC | 71.17 ± 0.71 | / | 63.92 ± 1.06 |
| INJ | 76.83 ± 0.71 | 48.58 ± 1.30 | 42.92 ± 1.53 |
| 3 | 85.08 ± 10.72 | / | 88.50 ± 6.84 |
| INJ + 3 | 76.08 ± 1.06 | 49.58 ± 1.30 | 46.50 ± 2.59 |
| 4 | 75.42 ± 3.18 | / | 90.83 ± 5.89 |
| INJ + 4 | 78.50 ± 2.59 | 49.33 ± 2.59 | 48.50 ± 1.41 |

Even in 6-hour recovery tissues samples, as expected TEER values were strongly reduced after mechanical injury, up to 49 OHM*cm², confirming the reproducibility of the injury procedure.

Positive control injured tissues sample (INJ) showed a −12% reduction of TEER after 6-hour post injury (from 48.58 to 42.92 OHM*cm²).

After 6-hour recovery, injured tissues treated with formulation No. 3 formulation No. 4 have maintained stable values of TEER.

FIG. 9b reports the TEER values measured on RHO treated with product No. 3 and No. 4 without injury. The data showed that TEER values comparable and higher to those found in the Negative Control sample.

Compared to basal value (t=0) for formulation No. 3 a difference of 4% has been shown and for formulation No. 4 an increase of absolute TEER value of 20% (from 75.42 to 90.83 OHM*cm²) has been recorded. Negative control has shown a TEER reduction of 10%.

LDH Release

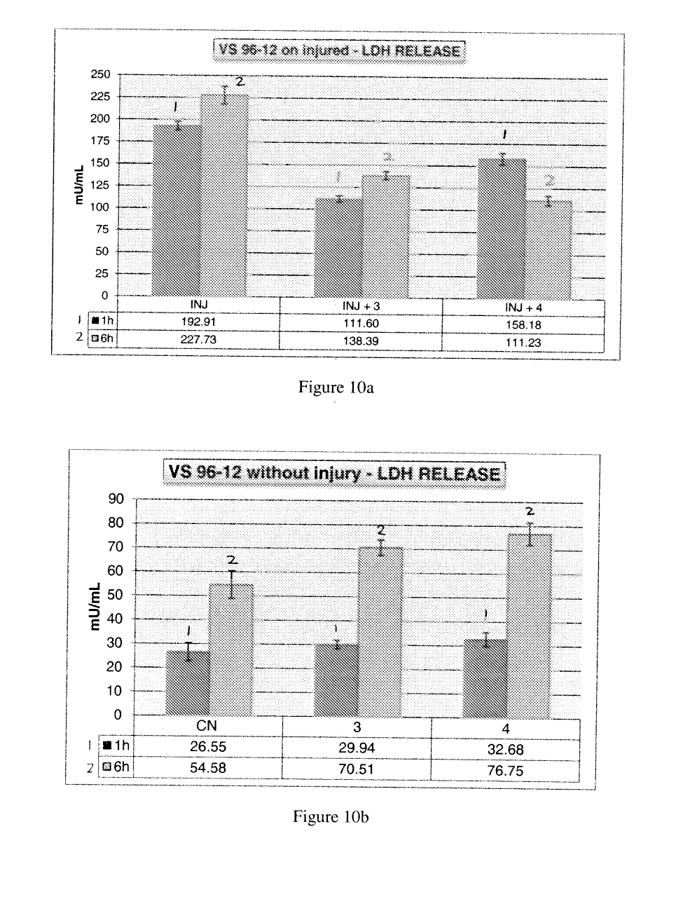

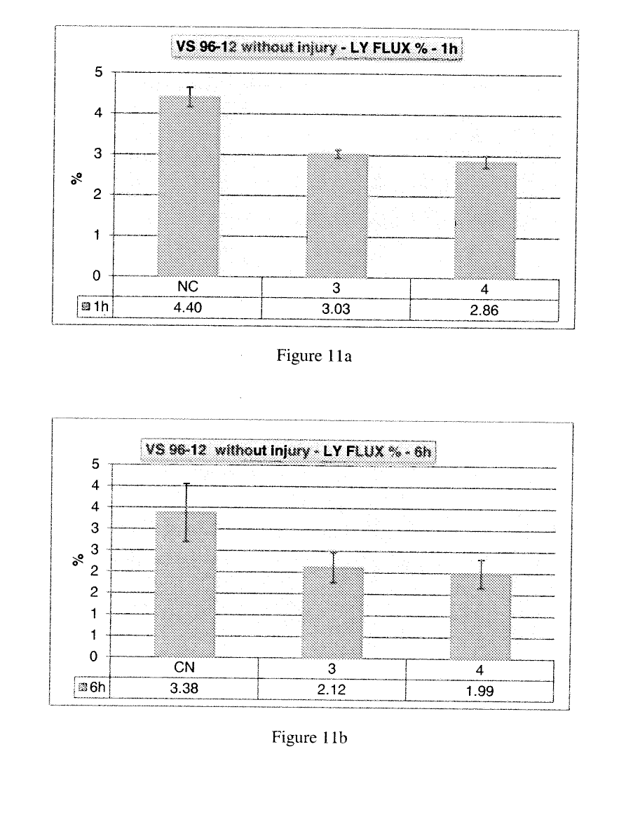

Lactate dehydrogenase (LDH) release in the culture medium was measured to detect the membrane integrity (Table 4, FIG. 10a, and FIG. 10b). This enzyme is normally present in the cytosol and cannot be measured extracellularly unless cell damage has occurred.

TABLE 4
LDH release after 10 min + 1 h/6 h, as depicted in FIG. 10a.

MEAN LDH mU/mL ± SD

| SAMPLE | 1 h | 6 h |
|---|---|---|
| NC | 26.55 ± 3.75 | 54.58 ± 5.76 |
| INJ | 192.91 ± 4.93 | 227.73 ± 10.08 |
| 3 | 29.94 ± 1.76 | 70.51 ± 3.23 |
| INJ + 3 | 111.60 ± 3.70 | 138.39 ± 4.51 |
| 4 | 32.68 ± 2.88 | 76.75 ± 4.68 |
| INJ + 4 | 158.18 ± 6.68 | 111.23 ± 5.35 |

Injured tissues samples (INJ) after 1 h and 6 h recovery showed a significantly high LDH release in medium, confirming that the damage induced by mechanical injury cannot be recovered during the post incubation (recovery) time.

Formulation No. 3 reduced LDH release compared to INJ sample at both exposure times, in particular after 1 h recovery. Formulation No. 4 showed a protective efficacy against injury induced damage in particular after 6 h recovery. See, FIG. 10b. Formulation No. 3 and No. 4 induced an LDH release comparable to Negative Control sample when applied on intact tissues after 1 h and slightly higher after 6 h.

Lucifer Yellow Assay

Lucifer yellow assay measures the modification of RHO permeability by using a fluorescent probe as marker of paracellular flux.

The LY assay is a very sensitive measure (more sensitive than the TEER measurement) of the tight junction structure and integrity: when the tight junctions are unbroken or reinforced, Lucifer Yellow has a very low permeability; if tight junctions are damaged, Lucifer Yellow flux increases.

The behavior of Formulations No. 3 and No. 4 when applied for 1 h (Table 5, FIG. 11a) and for 6 h (Table 5, FIG. 11b) on intact (non-injured tissues) showed a LY percentage flux even lower than the negative control (mean of 4%) at both exposures confirming the results previously obtained (Part-1). In fact, both formulations were able to reduce the paracellular flux suggesting a membrane barrier activity (reinforcement of the tight junction fence properties) and film forming properties.

TABLE 5
Lucifer Yellow flux after 10 min + 1 h/6 h, as depicted in FIG. 11.

LY FLUX % ± SD

| SAMPLE | 1 h | 6 h |
|---|---|---|
| NC | 4.40 ± 0.24 | 3.38 ± 0.68 |
| INJ | 25.45 ± 0.68 | 22.82 ± 1.05 |
| 3 | 3.03 ± 0.10 | 2.12 ± 0.35 |
| INJ + 3 | 13.95 ± 0.62 | 14.27 ± 2.51 |
| 4 | 2.86 ± 0.15 | 1.99 ± 0.33 |
| INJ + 4 | 17.36 ± 0.71 | 23.10 ± 0.37 |

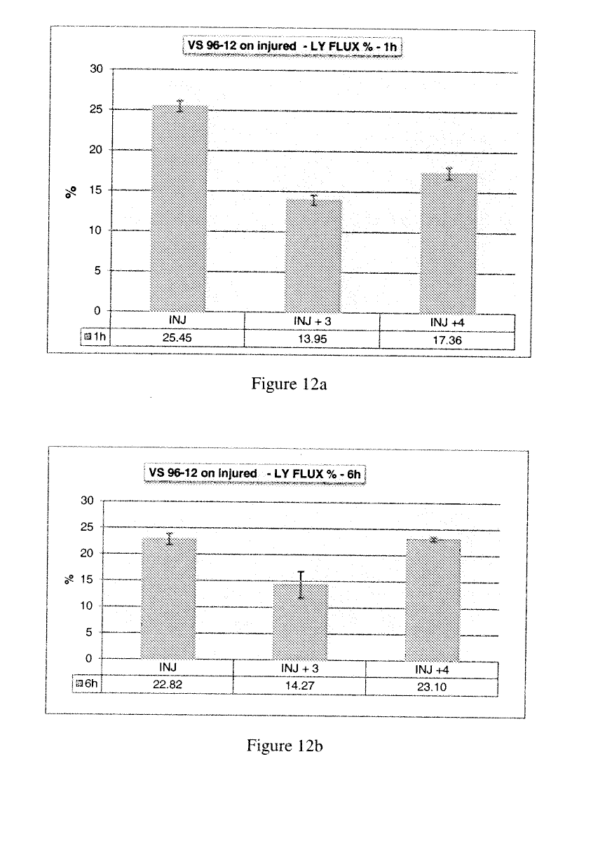

FIG. 12a and FIG. 12b show the results of LY flux. When injured tissues were treated with Formulation No. 3 paracellular flux was reduced down to 14% either after a short (1 hour) or long-lasting recovery (6 hours), indicating good recovery from the damage and a protective efficacy of the formulations.

Formulations No. 4 on injured tissues reduced LY passage after 1 h recovery reaching 17.36%, but it was not different from the INJ sample after 6 h recovery (23%).

The 2 sets of results demonstrate a protective efficacy and no harm on injured tissue treated with either product 3 or 4. Furthermore, protective efficacy was observed within one hour.

7. Conclusions

The present study has been conducted in order to assess membrane barrier function through the film forming and restoring efficacy of new formulations to be registered as Medical Device Class III containing a complex of “Cimetidine-N,O-carboxymethyl-chitosan”.

The test items have been tested in vitro using a reconstituted Human Oral Epithelium model (RHO) mechanically injured as biological model. Products have been tested compared to positive and negative controls and the parameters listed below have been evaluated after 10 minutes application on injured tissues followed by 1 h and 6 h recovery:

Trans-epithelial electrical resistance (TEER)

LDH release in culture media

Lucifer Yellow paracellular passage

As expected, the mechanical injury on RHO induced a strong reduction of TEER values, a significantly high LDH release in medium and a significantly high paracellular flux of Lucifer Yellow compared to the intact, non-injured sample.

According to the results obtained on injured RHO tissues, it is possible to conclude that Product Nos. 3 and 4 increase membrane barrier function due to film forming properties and have a protective and restoring efficacy of fence properties. This is evident due to the increase in TEER values, the reduction in LDH release into the medium at the one hour time point, and the decrease in LY paracellular flux, as compared to the positive (injured) control sample. Both products showed a very good tissue compatibility.

According to the LY paracellular flux results, injured tissue treated with Product No. 3 showed good recovery from the injury damage and protective efficacy after the short (1 h) and also the long (6 h) recovery periods. A significant flux reduction compared to control injured tissue was quantified (14.27% for product 3 compared to control values 22.82%).

Product No. 4 treatment showed a good and long lasting recovery from the injury damage, contributing to barrier membrane stability and with protective efficacy according to the reduction of LDH release seen at 1 hour and 6 hours, which suggests protective efficacy at membrane barrier level; slightly increase TEER values for both injured and not injured RHO; and the reduction of LY paracellular flux particularly in non-injured tissue.

This study has also confirmed results from Part 1 of the present study where Product 4 exhibited the highest efficacy as film forming and by modifying the membrane barrier, with a protective efficacy on non-injured tissue.

### Example 4

### Treatment of Inflammatory or Infectious Diseases

In a more direct test, hypo-fibrinogenemic mice were generated with repeated ancrod injection. Biomaterial implants in these mice failed to accumulate adherent phagocytes (unless the implants were pre-coated with murine fibrinogen). Subsequently it was found that following initial adsorption on hydrophobic biomaterial surfaces, fibrinogen undergoes conformational changes which expose previously occult epitopes on the gamma chain of fibrinogen (′PI′ (y190-202) and ‘P2’ (y377-392). These newly exposed epitopes are responsible for triggering the recruitment and activation of phagocytes, early events in the cascade of events involved in foreign body reactions. In mice, the initial recruitment of inflammatory cells to experimental implants is mediated by histamine. Mast cell deficient mice showed greatly diminished phagocyte accumulation on implants and administration of H1 and H2 receptor antagonists to normal mice substantially reduced phagocyte recruitment.

The implantation of medical devices often leads to foreign body reactions which are driven by the accumulation and activation of inflammatory cells. These acute inflammatory responses are very often followed by chronic inflammation and fibrosis. These reactions have been linked to the degradation and failure of many types of implants, including pacemaker leads, mammary prostheses, temporomandibular, and other joint implants. To improve the biocompatibility and safety of medical devices, intensive research efforts have been placed on the development of biomaterials with enhanced tissue compatibility. However, such efforts have been hindered by the lack of knowledge of the mechanisms governing foreign body reactions. In an attempt to understand these basic mechanisms, a series of experiments was carried out using a murine implantation model. Investigations revealed that following implantation, biomaterial surfaces rapidly become covered with a layer of host proteins. It was found that the adsorption and later “denaturation” of fibrinogen is the main factor in triggering biomaterial-mediated inflammatory responses in mice. Furthermore, it appeared that histamine release by mast cells in the vicinity of the implant was important in facilitating the recruitment of inflammatory cells inasmuch as both mast cell deficient mice and mice treated with histamine receptor antagonists had greatly reduced acute inflammatory responses to implanted biomaterials. However, it was not clear whether these processes, found to be important in mice, might be similarly crucial in humans with implanted biomaterials.

As a first test of this, we examined the importance of adsorbed fibrinogen in prompting foreign body reactions in human volunteers. As was true in the earlier murine model, plasma coated implants attracted significantly more phagocytes than did serum coated implants in humans. The process of inflammatory cell recruitment—in both mice and humans—likely involves the tendency of fibrinogen to adsorb and subsequently denature on the hydrophobic surfaces of biomedical polymers. The “denaturation” of adsorbed fibrinogen is particularly important inasmuch as it leads to the exposure of two epitopes on the fibrinogen gamma chain (PI and P2) which are normally occult in soluble fibrinogen. The exposure of these short sequences is required for both the adhesion and activation of phagocytes. Indeed, the degree of P1/P2 exposure engendered by different types of biomedical polymers predicts the extent of biomaterial-mediated inflammatory responses. Furthermore, it appears that the interaction between phagocytes and surface P1/P2 epitope is via the Mac-I (CD11b/CD18) integrin, which is upregulated on inflammatory cells recruited to the site of the implant.

Earlier animal studies also indicated that histamine, released from activated mast cells, is critical to the recruitment of phagocytes to both subcutaneous and intraperitoneal implants. This is in accord with numerous observations that biomedical implants trigger both edematous and hyperemic responses typically mediated by histamine. The pro-inflammatory effects of the released histamine evidently involve both H1 and H2 receptors. In the present experiments on humans, we observed that treatment with a combination of HI and H2 receptor antagonists reduced by more than 80% the accumulation of phagocytes on implant surfaces as was also true in the murine models. Since histamine exerts its action on capillary permeability and phagocyte transmigration through endothelial barrier, it is likely that histamine receptor antagonists diminish initial phagocyte recruitment probably through suppression of implant-mediated hyperemia and loosening of the endothelial barrier. This suggests that histamine antagonist administration shortly before and after the placement of biomedical implants in humans may lessen the phagocyte-mediated foreign body responses and later reactions such as fibrotic capsule formation around implanted medical devices. Johann Zdolsek, John W Eaton, and Liping Tang Histamine release and fibrinogen adsorption mediate acute inflammatory responses to biomaterial implants in humans. J Transl Med. 2007; 5: 31.

The enhancing effects of chitosan on activation of platelets and differentiation of osteoprogenitor cells have been demonstrated in vitro. Chitosan-collagen composites might induce in vivo new bone formation around pure titanium implant surfaces but different molecular weights of chitosan did not show significantly different effects on the osteoinductive potential of the test materials. Kung S, Devlin H, Fu E, Ho K Y, Liang S Y, Hsieh Y D. The osteoinductive effect of chitosan-collagen composites around pure titanium implant surfaces in rats. J. Periodontal Res. 2011 February; 46(1): 126-33. doi: 10.llll(j.1600-0765.2010.01322.x. Epub 2010 Nov. 26.

Chitosan is a biopolymer that exhibits osteoconductive, enhanced wound healing and antimicrobial properties which make it attractive for use as a bioactive coating to improve osseointegration of orthopedic and craniofacial implant devices. Coatings made from 91.2% de-acetylated chitosan were chemically bonded to titanium coupons via silane-glutaraldehyde molecules. The bond strength of the coatings was evaluated in mechanical tensile tests, and their dissolution and cytocompatibility were evaluated in vitro using cell-culture medium and UMR 106 osteoblastic cells, respectively. The results showed that the chitosan coatings were chemically bonded to the titanium substrate and that the bond strengths (1.5-1.8 MPa) were not affected by gas sterilization. However, the chitosan bond strengths were less than those reported for calcium-phosphate coatings. The gas-sterilized coatings exhibited little dissolution over 8 weeks in cell-culture solution, and the attachment and growth of the UMR 106 osteoblast cells was greater on the chitosan-coated samples than on the uncoated titanium. These results indicated that chitosan has the potential to be used as a biocompatible, bioactive coating for orthopedic and craniofacial implant devices. Bumgardner J D, Wiser R, Gerard P D, Bergin P, Chestnutt B, Marin M, Ramsey V, Elder S H, Gilbert J A. Chitosan: potential use as a bioactive coating for orthopaedic and craniofacial/dental implants. J Biomater Sci Polym Ed. (2003).

Chitosan has been shown to elicit a minor foreign body response similar to that of Gore-Tex, including thin fibrous encapsulation and early, yet non-persisting, activation of microglia/macrophages. Changes in the charge profile of chitosan occur over time, suggesting degradation of polymer chains. Chitosan is a relatively inert biomaterial that does not elicit an immune response, making it suitable for long-term spinal cord applications. It should be noted that chitosan becomes very brittle upon fixation, resulting in fracturing of chitosan into shards as an artifact of sectioning, as seen in. At 1 month, relatively thin fibrous encapsulation of chitosan was observed. Howard Kim, Charles H. Tator, Molly S. Shoichet, Chitosan implants in the rat spinal cord: Biocompatibility and biodegradation. Published online 4 Apr. 2011 in Wiley Online Library (wileyonlienlibrary.com). DOI: l 0.1002/jbm.a.33070.

As a result, there is a need in the art for stable, effective chitosan formulations that are suitable around implants. Several studies have shown the potential of chitosan in coated implants or localized delivery devices but several complications has been seen with the usage of chitosan around implant devices. Encapsulation of the material, difficulties with fixation of the material to the device, low strength in coated implants, dissolution over time, creation of soft tissue around implant, lack of immunological natural stimulation and action in inflammatory process.

The need in the art is a stable formulation and effective that can enhance the soft and hard tissues strength to foreign bodies around medical implants or dental implants in a localized application in temporal or permanent implant placements. Protection against opportunistic bacteria around implanted permanent or temporarily devices, consequently, with an anti-inflammatory response, bone preservation and stimulation of bone formation around implants. The combination of an H2 antagonist and a derivative water soluble chitosan can resolve this need without affecting the structure or form of dental implants as they have been proven successful and protecting the host with a non invasive local therapy as it is eliminated in a short period of time with a long protection.

A derivative water soluble chitosan is the solution specifically N,O-carboxymethyl-chitosan, in combination with an H2 antagonist can enhance and preserves the bone formation, reduces histamine levels consequently with a immunity natural protective response and reduces bacterial injuries as it has been seen in periodontal cases.

Sharing similarities with periodontitis in both the bacterial initiators and key immune components to those insults, the rate of disease progression and the severity of inflammatory signs for peri-implantitis has taken us to the conclusion that the compositions describe in European Patent Application No. EP 12194594.3-1456 and method of used described in U.S. provisional Patent application No. 61/790,394 could be used for the prevention and treatment (including delaying the progression) of oral inflammatory diseases around medical and specifically dental implants.

To the best of our knowledge, the molecular determinants of biomaterial-mediated acute inflammatory responses in humans. Biocompatibility could be improved by the delivery of combination compositions comprising an H2 antagonist with a chitosan derivative or water soluble chitosan as described in patents European Patent Application No. EP 12194594.3-1456.

Coating the surfaces of the implants with these compositions could reduce foreign body reactions to the implants. Furthermore, we confirmed the importance of histaminic responses in the pathogenesis of biomaterial-mediated inflammatory responses as described before around natural teeth. Histamine receptor antagonists can be used to limit both acute inflammatory responses and later fibrotic reactions (which may directly stem from the acute responses). Patients who might benefit from such antihistamine treatment include those being treated with joint implants, breast implants, tissue engineering implants and drug delivery devices and dental implants.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A composition comprising an H2 receptor antagonist and a water soluble chitosan derivative in admixture with a suitable carrier or excipient.

2. The composition of claim 1, wherein the H2 receptor antagonist is selected from the group consisting of cimetidine, ranitidine, famotidine, nizatidine, and a combination thereof.

3. (canceled)

4. The composition of claim 1, wherein the H2 receptor antagonist constitutes between 0.01% and 10% of the composition.

5. The composition of claim 1, wherein the water soluble chitosan derivative is a mucoadhesive polymer chitosan.

6. The composition of claim 1, wherein the water soluble chitosan derivative is N,O-carboxymethyl-chitosan.

7. The composition of claim 1, wherein the H2 receptor antagonist is cimetidine, and the water soluble chitosan derivative is N,O-carboxymethyl-chitosan.

8. The composition of claim 7, further comprising hyaluronic acid.

9. A composition comprising a host modulator and a water soluble chitosan derivative in admixture with suitable carriers and excipients.

10. The composition of claim 9, wherein the host modulator is selected from the group consisting of: a nonsteroidal anti-inflammatory drug (NSAID), a bisphosphonate, a tetracycline, a cytokine antagonist, a nitric oxide synthase inhibitor, an enamel matrix tetracycline, a growth factor, a bone morphogenetic protein, and a combination thereof.

11. (canceled)

12. The composition of claim 1, wherein the composition is suitable for topical oral administration.

13. (canceled)

14. The composition of claim 1, further comprising one or more active ingredients selected from the group consisting of a disinfectant of the oral cavity, a steroidal or non-steroidal anti-inflammatory agent, a wound healing agent, an analgesic agent, an antimicrobial agent, a host mediator, and an antihistamine.

15. The composition of claim 14, where the composition forms a protective film over one or more of: oral mucosa, skin, keratinized tissue, or epithelial basal cells.

16. The composition of claim 15, wherein the protective film stabilizes the oral mucosa by providing trans-epithelial electrical resistance.

17. The composition of claim 16, wherein the composition stabilizes the permeability of tissues by maintaining the integrity of a tight junction.

18. The composition of claim 17, wherein the stabilization maintains the integrity of oral mucosa.

19-27. (canceled)

28. A method of treating or preventing gingival or periodontal pathology characterized by inflammation and pain comprising administering an effective amount of the composition of claim 1.

29-31. (canceled)

32. A method of preventing a complication of systemic disease associated with oral inflammatory disease comprising administering a safe and effective amount of the composition of claim 1.

33-46. (canceled)

47. A method of treating or preventing gingival or periodontal pathology around implantable medical devices and or dental implant characterized by inflammation and pain comprising administering an effective amount of the composition of claim 1.

48-59. (canceled)

60. The composition of claim 1, wherein the composition is suitable for coating on an implantable device.

61-80. (canceled)

81. The composition of claim 1, in the form of a topical composition for the application to the skin, or vaginal or rectal mucous membrane.

82-89. (canceled)
